SEQUENCE LISTING (15 sequences)

SEQ ID NO: 1 - protein, 5 aa, Artificial Sequence
  source: /note=“Description of Artificial Sequence Synthetic peptide”
SYYIH

SEQ ID NO: 2 - protein, 17 aa, Artificial Sequence
  source: /note=“Description of Artificial Sequence Synthetic peptide”
  VARIANT (16)..(16): /replace=“Gln”
  MISC_FEATURE (1)..(17): /note=“Variant residues given in the sequence have no preference with respect to those in the annotations for variant positions”
VIYPGNDDISYNQKFKG

SEQ ID NO: 3 - protein, 9 aa, Artificial Sequence
  source: /note=“Description of Artificial Sequence Synthetic peptide”
EVRLRYFDV

SEQ ID NO: 4 - protein, 17 aa, Artificial Sequence
  source: /note=“Description of Artificial Sequence Synthetic peptide”
KSSQSVFFSSSQKNYLA

SEQ ID NO: 5 - protein, 7 aa, Artificial Sequence
  source: /note=“Description of Artificial Sequence Synthetic peptide”
WASTRES

SEQ ID NO: 6 - protein, 8 aa, Artificial Sequence
  source: /note=“Description of Artificial Sequence Synthetic peptide”
HQYLSSRT

SEQ ID NO: 7 - protein, 118 aa, Artificial Sequence
  source: /note=“Description of Artificial Sequence Synthetic polypeptide”
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 8 - protein, 113 aa, Artificial Sequence
  source: /note=“Description of Artificial Sequence Synthetic polypeptide”
NIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKR

SEQ ID NO: 9 - protein, 118 aa, Artificial Sequence
  source: /note=“Description of Artificial Sequence Synthetic polypeptide”
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 10 - protein, 113 aa, Artificial Sequence
  source: /note=“Description of Artificial Sequence Synthetic polypeptide”
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 11 - protein, 447 aa, Artificial Sequence
  source: /note=“Description of Artificial Sequence Synthetic polypeptide”
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 219 aa, Artificial Sequence
  source: /note=“Description of Artificial Sequence Synthetic polypeptide”
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 321 aa, Homo sapiens
MAAAEAGGDDARCVRLSAERAQALLADVDTLLFDCDGVLWRGETAVPGAPEALRALRARGKRLGFITNNSSKTRAAYAEKLRRLGFGGPAGPGASLEVFGTAYCTALYLRQRLAGAPAPKAYVLGSPALAAELEAVGVASVGVGPEPLQGEGPGDWLHAPLEPDVRAVVVGFDPHFSYMKLTKALRYLQQPGCLLVGTNMDNRLPLENGRFIAGTGCLVRAVEMAAQRQADIIGKPSRFIFDCVSQEYGINPERTVMVGDRLDTDILLGATCGLKTILTLTGVSTLGDVKNNQESDCVSKKKMVPDFYVDSIADLLPALQG

SEQ ID NO: 14 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 15 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

(19) United States
(12) Patent Application Publication
(10) Pub. No.: US 2020/0261470 A1
(43) Pub. Date: Aug. 20, 2020

(54) COMBINATION TREATMENT WITH ANTIBODY-DRUG CONJUGATES AND PARP INHIBITORS

(71) Applicants: IMMUNOGEN, INC., Waltham, MA (US); HEALTH RESEARCH, INC., Buffalo, MA (US)

(72) Inventors: Eunice Sue Wang, Williamsville, NY (US); Scott Michael Portwood, Orchard Park, NY (US); Russell Walker, Dedham, MA (US)

(21) Appl. No.: 16/346,950

(22) PCT Filed: Nov. 1, 2017
(86) PCT No.: PCT/US2017/059483

§ 371 Date: May 2, 2019

Related U.S. Application Data
(60) Provisional application No. 62/416,383, filed on Nov. 2, 2016.

(51) Int. Cl. A61K 31/5517; A61K 47/68; A61K 39/395; A61K 31/502; A61K 31/5025; A61K 31/454; A61K 31/4184; A61K 31/55; A61P 35/02

(52) U.S. Cl. CPC A61K 31/5517; A61K 47/6803; A61K 39/3955; A61K 31/502; A61P 35/02; A61K 31/454; A61K 31/4184; A61K 31/55; A61K 31/5025

(57) ABSTRACT

The present invention provides a method of treating a cancer in a subject comprising administering to the subject an effective amount of a CD33-targeted antibody-drug conjugate (ADC) and an effective amount of a poly-ADP ribose polymerase (PARP) inhibitor. Also provided are pharmaceutical compositions comprising an effective amount of a CD33-targeted ADC and an effective amount of a PARP inhibitor.

### RELATED APPLICATIONS

This application is a 35 U.S.C. § 371 national stage filing of International Application No. PCT/US2017/059483, filed on Nov. 1, 2017, which claims priority to U.S. Provisional Patent Application No. 62/416,383, filed on Nov. 2, 2016. The entire contents of each of the foregoing applications are expressly incorporated herein by reference.

### SEQUENCE LISTING

The instant application contains a Sequence Listing which has been submitted electronically in ASCII format and is hereby incorporated by reference in its entirety. Said ASCII copy, created on Oct. 26, 2017, is named 121162-03720_SL.txt and is 27,280 bytes in size.

### BACKGROUND OF THE INVENTION

Acute myeloid leukemia (AML) is associated with the accumulation of abnormal blast cells in bone marrow. Acute myeloid leukemia (AML) is one of the most common types of leukemia among adults. In the United States alone, over 18,000 new cases of AML are identified each year, and more than 10,000 deaths are associated with AML. Despite high initial response rates to chemotherapy, many acute myeloid leukemia (AML) patients fail to achieve complete remission. In fact, the majority of patients with AML relapse within 3-5 years from diagnosis.

The leukocyte differentiation antigen CD33 is a 364 amino acid transmembrane glycoprotein with sequence homology to members of the sialoadhesin family, including myelin-associated glycoprotein and CD22, as well as sialoadhesin itself (S. Peiper, 2002, Leucocyte Typing VII, White Cell Differentiation, Antigens, Proceedings of the Seventh International Workshop and Conference, Oxford University Press, p. 777).

Expression of CD33 appears to be highly specific to the hematopoietic compartment, with strong expression by myeloid precursor cells (S. Peiper, 2002). It is expressed by myeloid progenitor cells such as CFU-GEMM, CFU-GM, CFU-G and BFU-E, monocytes/macrophages, granulocyte precursors such as promyelocytes and myelocytes although with decreased expression upon maturation and differentiation, and mature granulocytes though with a low level of expression (S. Peiper, 2002). Anti-CD33 monoclonal antibodies have shown that CD33 is expressed by clonogenic, acute myelogenous leukemia (AML) cells in greater than 80% of human cases (LaRussa, V. F. et al., 1992, Exp. Hematol. 20:442-448). In contrast, pluripotent hematopoietic stem cells that give rise to “blast colonies” in vitro (Leary, A. G. et al., 1987, Blood 69:953) and that induce hematopoietic long-term marrow cultures (Andrews R. G. et al., 1989, J. Exp. Med. 169:1721; Sutherland, H. J. et al., 1989, Blood 74:1563) appear to lack expression of CD33.

Due to the selective expression of CD33, antibody drug conjugates (hereinafter “ADCs”) that combine cytotoxic drugs with monoclonal antibodies that specifically recognize and bind CD33 have been proposed for use in selective targeting of AML cells. Such therapies are expected to leave stem cells and primitive hematopoietic progenitors unaffected. Recently, a CD33-targeted ADC utilizing a novel DNA alkylator, DGN462, which comprises an indolino-benzodiazepine dimer containing a monoimine moiety has been reported (see, for example, U.S. Pat. Nos. 8,765,740, 8,889,669, 9,169,272, and 9,434,748) that shows anti-cancer activity in in vitro and in vivo hematologic cancer models. While this ADC shows great promise, there is still a need for improved methods for using the ADC in treating patients suffering from cancer, in particular hematologic cancers, such as, AML.

### SUMMARY OF THE INVENTION

Poly-ADP ribose polymerase (PARP) inhibitors are known to be used in treating solid tumors, particularly solid tumors with DNA repair defects. Their efficacy in treating hematologic cancers has not been well established. It has now been surprisingly found that the combination of a CD33-targeted ADC containing an indolino-benzodiazepine dimer cytotoxic payload with a PARP inhibitor has synergistic effects against leukemia cells both in vitro and in vivo as compared with the ADC alone and the PAPR inhibitor alone. For example, a synergistic reduction in cancer cell proliferation was observed when human CD33+ acute myeloid leukemia cells (HEL, MV4-11, and HL60) were treated with the combination of a CD33-targeted ADC, IMGN779, and the PARP inhibitor, olaparib (See Example 1). Additionally, the combination of IMGN779 with olaparib i) further decreased tumor burden in an acute myeloid leukemia xenograft animal model compared with either drug alone (See Example 2); and ii) was effective in inhibiting colony formation of primary cells from patients with relapsed/refractory acute myeloid leukemia characterized by complex karyotype or FLT-3 mutations (See Example 3). Based on these surprising findings, the present invention provides methods of treating a cancer, e.g., a hematologic cancer such as AML, with a combination of a CD33-targeted ADC containing an indolino-benzodiazepine dimer cytotoxic payload and a PARP inhibitor described herein. Also disclosed are pharmaceutical compositions comprising the CD33-targeted ADC containing an indolino-benzodiazepine dimer cytotoxic payload and the PARP inhibitor.

One embodiment of the invention is a method for treating a cancer in a subject. In one embodiment, the cancer is selected from acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), basic plasmacytoid DC neoplasm (BPDCN) leukemia, non-Hodgkin lymphomas (NHL), mantle cell lymphoma, and Hodgkin's leukemia (HL). In another embodiment, the cancer is chemotherapy sensitive. In another embodiment, the cancer is chemotherapy resistant. In another embodiment, the cancer is acute myeloid leukemia (AML). In yet another embodiment, the AML is refractory or relapse acute myeloid leukemia. In yet tanother another embodiment, the AML is characterized by overexpression of P-glycoprotein, overexpression of EVI1, a p53 alteration, DNMT3A mutation, FLT3 internal tandem duplication, a complex karyotype, decreased expression in BRCA1, BRCA2, or PALB2, or mutations in BRCA1, BRCA2, or PALB2. The method comprises the steps of administering to the subject an effective amount of a PARP inhibitor and an effective amount of an ADC of Formula (I):

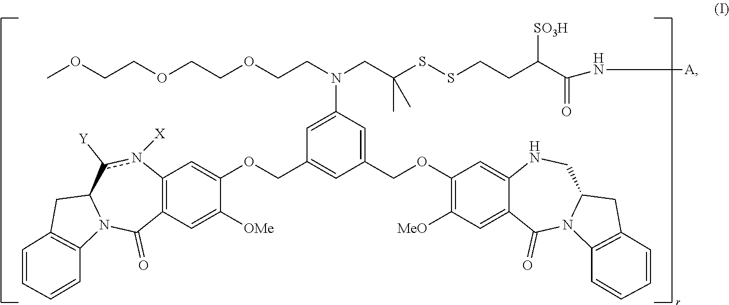

or a pharmaceutically acceptable salt thereof. The double line between [symbol] and C represents either a single bond or a double bond, provided that when it is a double bond, X is absent and Y is hydrogen; and when it is a single bond, X is hydrogen and Y is —SO₃H. The term “A” is an antibody or antigen-binding fragment that binds to CD33. Alternatively, “A” is an antibody or antigen-binding fragment that specifically binds to CD33 comprising a heavy chain variable region (VH) complementary determining region (CDR)1 sequence of SEQ ID NO:1, a VH CDR2 sequence of SEQ ID NO:2, and a VH CDR3 sequence of SEQ ID NO:3, and a light chain variable region (VL) CDR1 sequence of SEQ ID NO:4, a VL CDR2 sequence of SEQ ID NO:5, and a VL CDR3 sequence of SEQ ID NO:6. The term “r” is an integer from 1 to 10.

In one embodiment, the antibody or antigen-binding fragment thereof comprises a heavy chain variable region comprising an amino acid sequence having at least 95% identity to the amino acid sequence of SEQ ID NO:7 or 9. In another embodiment, the antibody or antigen-binding fragment thereof comprises a light chain variable region comprising an amino acid sequence having at least 95% identity to the amino acid sequence of SEQ ID NO:8 or 10. In another embodiment, the antibody or antigen-binding fragment thereof comprises a heavy chain variable region comprising the sequence of SEQ ID NO:9 and a light chain variable region comprising the sequence of SEQ ID NO:10. In another embodiment, the antibody is huMy9-6. In yet another embodiment, the antibody is a CDR-grafted or resurfaced antibody.

ADC1, ADC2, IMGN779 (defined below) and pharmaceutically acceptable salts thereof, are specific examples of ADCs that can be used in the disclosed methods of treatment.

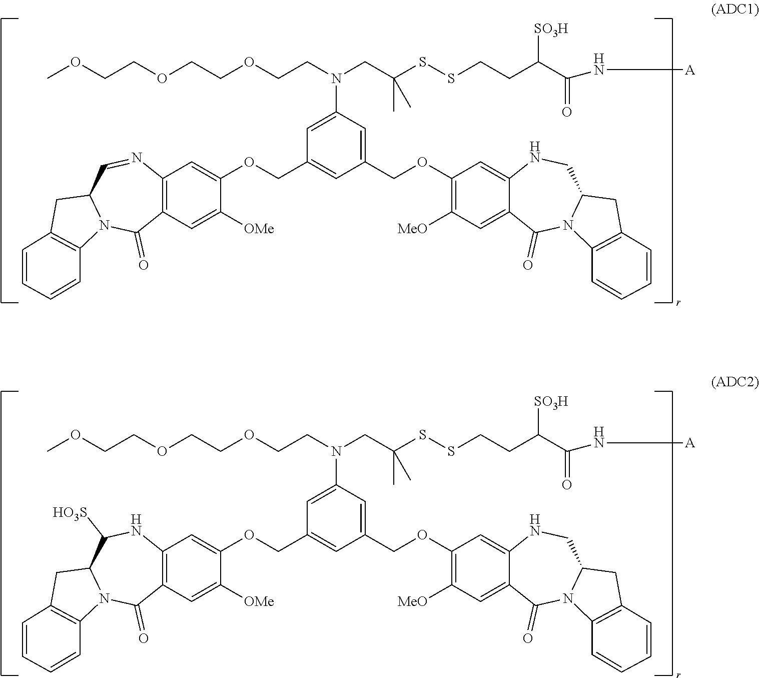

“A” is as defined for Formula (I). The term “r” is an integer from 1 to 10. Methods of preparing ADC1, ADC2, and IMGN779 are provided in U.S. Pat. Nos. 8,765,740 and 9,353,127, the entire teachings of which are incorporated herein by reference.

Pharmaceutically acceptable salts are those which are suitable for use in humans and animals without undue toxicity, irritation, and allergic response. Examples for suitable salts for the ADC of Formula (I), ADC1, ADC2, and IMGN779 are disclosed in U.S. Pat. No. 8,765,740, the entire teachings of which are incorporated herein by reference. In one embodiment, the pharmaceutically acceptable salt for the ADCs of Formula (I), ADC1, ADC2, and IMGN779 is the sodium or potassium salt.

Another embodiment of the invention is a pharmaceutical compositions comprising: i) an effective amount of a PARP inhibitor; ii) an effective amount of an antibody-drug conjugate of Formula (I), ADC1, ADC2, IMGN779 or a pharmaceutically acceptable salt thereof; and iii) a pharmaceutically acceptable carrier or diluent. In one embodiment, the pharmaceutically acceptable salt for the ADCs of Formula (I), ADC1, ADC2, and IMGN779 is the sodium or potassium salt.

Another embodiment of the invention is an antibody-drug conjugate of Formula (I), ADC1, ADC2, IMGN779 or a pharmaceutically acceptable salt thereof for treating a subject with cancer, in combination with a PARP inhibitor. In one embodiment, the pharmaceutically acceptable salt for the ADCs of Formula (I), ADC 1 ADC 2, and IMGN779 is the sodium or potassium salt. In one embodiment the cancer is selected from acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), basic plasmacytoid DC neoplasm (BPDCN) leukemia, non-Hodgkin lymphomas (NHL), mantle cell lymphoma, and Hodgkin's leukemia (HL). In another embodiment, the cancer is chemotherapy sensitive. In another embodiment, the cancer is chemotherapy resistant. In another embodiment, the cancer is acute myeloid leukemia (AML). In yet another embodiment, the AML is refractory or relapse acute myeloid leukemia. In yet tanother another embodiment, the AML is characterized by overexpression of P-glycoprotein, overexpression of EVI1, a p53 alteration, DNMT3A mutation, FLT3 internal tandem duplication, a complex karyotype, decreased expression in BRCA1, BRCA2, or PALB2, or mutations in BRCA1, BRCA2, or PALB2.

Yet another embodiment of the invention is the use an antibody-drug conjugate of Formula (I), ADC1, ADC2, IMGN779 or a pharmaceutically acceptable salt thereof for the manufacture of a medicament for treating a subject with cancer in combination with a PARP inhibitor. In one embodiment, the pharmaceutically acceptable salt for the ADCs of Formula (I), ADC1, ADC2, and IMGN779 is the sodium or potassium salt. In another embodiment the cancer is selected from acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome (MDS), basic plasmacytoid DC neoplasm (BPDCN) leukemia, non-Hodgkin lymphomas (NHL), mantle cell lymphoma, and Hodgkin's leukemia (HL). In another embodiment, the cancer is chemotherapy sensitive. In another embodiment, the cancer is chemotherapy resistant. In another embodiment, the cancer is acute myeloid leukemia (AML). In yet another embodiment, the AML is refractory or relapse acute myeloid leukemia. In yet another another embodiment, the AML is characterized by overexpression of P-glycoprotein, overexpression of EVI1, a p53 alteration, DNMT3A mutation, FLT3 internal tandem duplication, a complex karyotype, decreased expression in BRCA1, BRCA2, or PALB2, or mutations in BRCA1, BRCA2, or PALB2.

### Definitions

By “IMGN779” is meant a CD33-targeted ADC comprising the huMy9-6 or Z4681A antibody (i.e., an antibody comprising the heavy chain CDR1-3 having the sequence of SEQ ID NOs:1-3, respectively and the light chain CDR1-3 having the sequence of SEQ ID NOs:4-6; an antibody comprising the heavy chain variable region having the sequence of SEQ ID NO:9 and a light chain variable region having the sequence of SEQ ID NO:10; or an antibody comprising the heavy chain sequence having the sequence of SEQ ID NO:11 and the light chain sequence having the sequence of SEQ ID NO:12), conjugated to DGN462, via a cleavable disulfide linker. IMGN779 may be represented as ADC3 as depicted below:

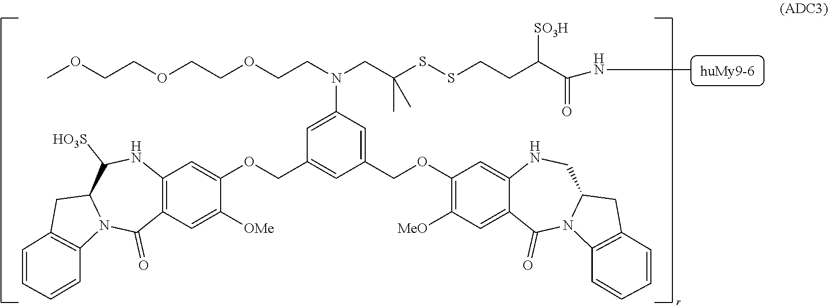

or a pharmaceutically acceptable salt thereof; or IMGN779 may also be represented as ADC4 as depicted below:

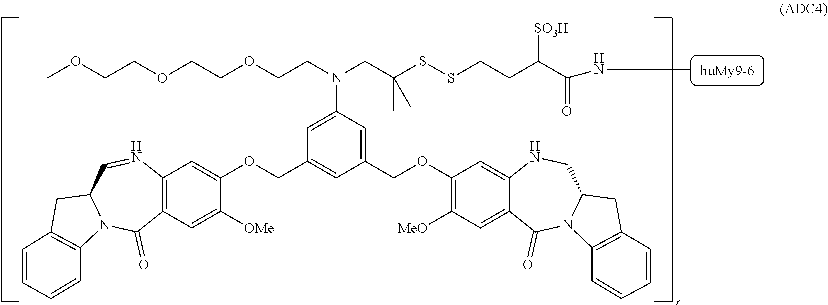

or a pharmaceutically acceptable salt thereof; or IMGN779 can be a combination of ADC3 and ADC4 or pharmaceutically acceptable salts thereof.

By “P-glycoprotein” is meant a polypeptide or fragment thereof having at least about 85% amino acid sequence identity to the human sequence provided at NCBI Accession No. NP_001035830 and conferring multi-drug resistance on a cell in which it is expressed. The sequence of an exemplary human P-glycoprotein is provided below:

| | |
|---|---|
| 1 | maaaeaggdd arcvrlsaer aqalladvdt llfdcdgvlw rgetavpgap ealralrarg |
| 61 | krlgfitnns sktraayaek lrrlgfggpa gpgaslevfg tayctalylr qrlagapapk |
| 121 | ayvlgspala aeleavgvas vgvgpeplqg egpgdwlhap lepdvravvv gfdphfsymk |
| 181 | ltkalrylqq pgcllvgtnm dnrlplengr fiagtgclvr avemaaqrqa diigkpsrfi |
| 241 | fdcvsqeygi npertvmvgd rldtdillga tcglktiltl tgvstlgdvk nnqesdcvsk |
| 301 | kkmvpdfyvd siadllpalq g |

By “CD33 protein” is meant a polypeptide or fragment thereof having at least about 85% amino acid sequence identity to the human sequence provided at NCBI Accession No. CAD36509 and having anti-CD33 antibody binding activity. An exemplary human CD33 amino acid sequence is provided below:

| | |
|---|---|
| 1 | mplllllpll wagalamdpn fwlqvqesvt vqeglcvlvp ctffhpipyy dknspvhgyw |
| 61 | fregaiisrd spvatnkldq evqeetqgrf rllgdpsrnn cslsivdarr rdngsyffrm |
| 121 | ergstkysyk spqlsvhvtd lthrpkilip gtlepghskn ltcsyswace qgtppifswl |
| 181 | saaptslgpr tthssvliit prpqdhgtnl tcqvkfagag vttertiqln vtyvpqnptt |
| 241 | gifpgdgsgk qetragvvhg aiggagvtal lalclcliff ivkthrrkaa rtavgrndth |
| 301 | pttgsaspkh qkksklhgpt etsscsgaap tvemdeelhy aslnfhgmnp skdtsteyse |
| 361 | vrtq |

By “FLT3 protein,” “FLT3 polypeptide,” “FLT3,” “FLT-3 Receptor,” or “FLT-3R” is meant a polypeptide or fragment thereof having at least about 85%, 90%, 95%, 99% or 100% amino acid sequence identity to the human sequence of FLT3 tyrosine kinase receptor, also referred to as FLK-2 and STK-1, provided at NCBI Accession No. NP_004110 and having tyrosine kinase activity, including receptor tyrosine kinase activity. In one embodiment the FLT3 amino acid sequence is the human FLT3 amino acid sequence provided below:

| | |
|---|---|
| 1 | mpalardggq lpllvvfsam ifgtitnqdl pvikcvlinh knndssvgks ssypmvsesp |
| 61 | edlgcalrpq ssgtvyeaaa vevdvsasit lqvlvdapgn isclwvfkhs slncqphfdl |
| 121 | qnrgvvsmvi lkmtetqage yllfiqseat nytilftvsi rntllytlrr pyfrkmenqd |
| 181 | alvcisesvp epivewvlcd sqgesckees pavvkkeekv lhelfgtdir ccarnelgre |
| 241 | ctrlftidln qtpqttlpql flkvgeplwi rckavhvnhg fgltwelenk aleegnyfem |
| 301 | stystnrtmi rilfafvssv arndtgyytc ssskhpsqsa lvtivekgfi natnssedye |
| 361 | idqyeefcfs vrfkaypqir ctwtfsrksf pceqkgldng ysiskfcnhk hqpgeyifha |
| 421 | enddaqftkm ftlnirrkpq vlaeasasqa scfsdgyplp swtwkkcsdk spncteeite |
| 481 | gvwnrkanrk vfgqwvssst lnmseaikgf lvkccaynsl gtscetilln spgpfpfiqd |
| 541 | nisfyatigv cllfivvltl lichkykkqf ryesqlqmvq vtgssdneyf yvdfreyeyd |
| 601 | lkwefprenl efgkvlgsga fgkvmnatay gisktgvsiq vavkmlkeka dsserealms |
| 661 | elkmmtqlgs henivnllga ctlsgpiyli feyccygdll nylrskrekf hrtwteifke |
| 721 | hnfsfyptfq shpnssmpgs revqihpdsd qisglhgnsf hsedeieyen qkrleeeedl |
| 781 | nvltfedllc fayqvakgme flefkscvhr dlaarnvlvt hgkvvkicdf glardimsds |
| 841 | nyvvrgnarl pvkwmapesl fegiytiksd vwsygillwe ifslgvnpyp gipvdanfyk |
| 901 | liqngfkmdq pfyateeiyi imqscwafds rkrpsfpnlt sflgcqlada eeamyqnvdg |
| 961 | rvsecphtyq nrrpfsremd lgllspqaqv eds |

By “FLT3-ITD” is meant a FLT3 polypeptide having internal tandem duplication(s) including but not limited to simple tandem duplication(s) and/or tandem duplication(s) with insertion. In various embodiments, FLT3 polypeptides having internal tandem duplications are activated FLT3 variants (e.g., constitutively autophosphorylated). In some embodiments, the FLT3-ITD includes tandem duplications and/or tandem duplication(s) with insertion in any exon or intron including, for example, exon 11, exon 11 to intron 11, and exon 12, exon 14, exon 14 to intron 14, and exon 15. The internal tandem duplication mutation (FLT3-ITD) is the most common FLT3 mutation, present in about 20-25% of AML cases. Patients with FLT3-ITD AML have a worse prognosis than those with wild-type (WT) FLT3, with an increased rate of relapse and a shorter duration of response to chemotherapy.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “specifically binds” is meant an antibody or fragment thereof that recognizes and binds a polypeptide of interest, but which does not substantially recognize and bind other molecules in a sample, for example, a biological sample, which naturally includes a polypeptide of the invention.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

“Effective amount” means that amount of ADC or PARP inhibitor that elicits the desired biological response in a subject. Such response includes alleviation of the symptoms of the disease or disorder being treated, inhibition or a delay in the recurrence of symptom of the disease or of the disease itself, an increase in the longevity of the subject compared with the absence of the treatment, or inhibition or delay in the progression of symptom of the disease or of the disease itself. Toxicity and therapeutic efficacy of the ADC or PARP inhibitor can be determined by standard pharmaceutical procedures in cell cultures and in experimental animals. The effective amount of the ADC or PARP inhibitor to be administered to a subject will depend on the stage, category and status of the multiple myeloma and characteristics of the subject, such as general health, age, sex, body weight and drug tolerance. The effective amount of the ADC or PARP inhibitor to be administered will also depend on administration route and dosage form. Dosage amount and interval can be adjusted individually to provide plasma levels of the active compound that are sufficient to maintain desired therapeutic effects.

The terms “treatment,” “treat,” and “treating” refer to reversing, alleviating, or inhibiting the progress of a cancer, or one or more symptoms thereof, as described herein.

The terms “administer”, “administering”, “administration”, and the like, as used herein, refer to methods that may be used to enable delivery of the ADCs and PARP inhibitors to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa. In one aspect, the ADC and/or PARP inhibitor are administered intravenously.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A shows HEL CD33+ AML cells treated with 500 pM IMGN779, 50 μM olaparib (Ola), or 500 pM IMGN779+50 μM olaparib and the effects on proliferation as measured by WST-8 reagent.

FIG. 1B shows the synergy/additive effects for the combination of IMGN779+ olaparib in HEL cells as calculated using Compusyn software. Data points below the line represent synergy between the drug pairs.

FIG. 2A shows MV4-11 CD33+ AML cells treated with 750 pM IMGN779, 12 μM olaparib (Ola), or 750 pM IMGN779+12 μM olaparib and the effects on proliferation as measured by WST-8 reagent.

FIG. 2B shows the synergy/additive effects for the combination of IMGN779+ olaparib in MV4-11 cells as calculated using Compusyn software. Data points below the line represent synergy between the drug pairs.

FIG. 3A shows HL60 CD33+ AML cells treated with 25 pM IMGN779, 10 μM olaparib (Ola), or 25 pM IMGN779+10 μM olaparib and the effects on proliferation as measured by WST-8 reagent.

FIG. 3B shows the synergy/additive effects for the combination of IMGN779+ olaparib in HL60 cells as calculated using Compusyn software. Data points below the line represent synergy between the drug pairs.

FIG. 4 shows cell viability and cell cycle effects in HEL cells treated with 500 pM IMGN779, 50 μM olaparib, or 500 pm IMGN779+50 μM olaparib as assessed by flow cytometry.

FIG. 5 shows percentage apoptosis in HEL cells treated with 500 pM IMGN779, 50 μM olaparib, or 500 pm IMGN779+50 μM olaparib.

FIG. 6A shows HEL cells treated with graded concentrations of olaparib and the effects on cell death after DNA-damaging radiation exposure at 0.5 Gy radiation.

FIG. 6B shows HEL cells treated with graded concentrations of olaparib and the effects on cell death after DNA-damaging radiation exposure at 0.75 Gy radiation.

FIG. 7A shows the anti-leukemic activity of IMGN779 at varying concentrations (30 μg/kg, 60 μg/kg, and 100 μg/kg (by payload)) in a systemic HEL AML xenograft model.

FIG. 7B shows leukemic burden on day 14 in mice after dosing with vehicle or IMGN779 at varying concentrations (30 μg/kg, 60 μg/kg, and 100 μg/kg (by payload)).

FIG. 7C shows overall survival in a systemic HEL AML xenograft model treated with varying concentrations of IMGN779 (30 μg/kg, 60 μg/kg, and 100 μg/kg (by payload)).

FIG. 8A shows the anti-leukemic activity of IMGN779 (15 μg/kg), olaparib (100 mg/kg), and IMGN779 (15 μg/kg)+ olaparib (100 mg/kg) in a systemic HEL AML xenograft model.

FIG. 8B shows leukemic burden on day 22 in mice after dosing with vehicle, IMGN779 alone (15 μg/kg), olaparib alone (100 mg/kg), or combination treatment with IMGN779 ((15 μg/kg)+ olaparib (100 mg/kg).

FIG. 8C shows overall survival in a systemic HEL AML xenograft model treated with IMGN779 (15 μg/kg), olaparib (100 mg/kg), and IMGN779 ((15 μg/kg)+ olaparib (100 mg/kg)).

FIG. 9A shows results from CFU assays quantified 15 days after plating using a Spot-RT3 camera mounted to an inverted microscope with SPOT-Basic imaging software. A representative sample for each condition was captured and triplicate wells were averaged and reported (+/− standard deviation).

FIG. 9B shows the effects of 1 μM olaparib, 10 μM IMGN779, and olaparib (1 μM)+ IMGN779 (10 pM) on colony formation of cells from bone marrow samples of patients with relapsed/refractory AML. Cells were incubated for 15 days at 37° C. then quantified using a Spot-RT3 camera mounted to an inverted microscope with SPOT-Basic imaging software. Representative sample images for each treatment condition are shown.

FIGS. 10A and 10B show proliferation of (A) HEL-luc and (B) HL60 cell lines after treatment with rucaparib, velparib, niraparib, talazoparib, and olaparib at various concentrations.

FIGS. 11A, 11B and 11C show HEL-luc cells treated with: 800 pM IMGN779, 0.8 μM talazoparib (Tal), or 800 pM IMGN779+0.8 μM Tal (FIG. 11A); 800 pM IMGN779, 0.8 μM olaparib (Ola), 800 pM IMGN779+0.8 μM Ola (FIG. 11B); 800 pM IMGN779, 0.8 μM niraparib (Nir), or 800 pM IMGN779+0.8 μM Nir (FIG. 11C), and the effects on proliferation as measured by WST-8 reagent.

FIGS. 12A and 12B show the synergy/additive effects for the combination of IMGN779+ Niraparib (FIG. 12A) and the combination of IMGN779+ Talazoparib (FIG. 12B) in HEL-luc cells as calculated using Compusyn software. Data points below the line represent synergy between the drug pairs.

FIGS. 13A, 13B and 13C show percentage apoptosis in HEL-luc cells treated with 800 pM IMGN779, 0.8 μM olaparib, or 800 pm IMGN779+0.8 μM olaparib (FIG. 13A); 800 pM IMGN779, 0.8 μM talaparib, or 800 pm IMGN779+0.8 μM talaparib (FIG. 13B); and 800 pM IMGN779, 0.8 μM niraparib, or 800 pm IMGN779+0.8 μM niraparib (FIG. 13C), as measured by flow cytometry.

FIGS. 14A, 14B and 14C show cell viability and cell cycle effects in HEL-luc cells treated with 800 pM IMGN779, 0.8 μM talaparib, or 800 pm IMGN779+0.8 μM talaparib (FIG. 14A); 800 pM IMGN779, 0.8 μM olaparib, or 800 pm IMGN779+0.8 μM olaparib (FIG. 14B); and 800 pM IMGN779, 0.8 μM niraparib, or 800 pm IMGN779+0.8 μM niraparib (FIG. 14C), as assessed by flow cytometry.

FIGS. 15A, 15B and 15C show extent of DNA damage as measured by % positive for phosphorylated H2AX staining in HEL-luc cells treated with: 800 pm IMGN779, 0.8 μM talaparib, or 800 pm IMGN779+0.8 μM talaparib (FIG. 15A); 800 pM IMGN779, 0.8 μM olaparib, or 800 pm IMGN779+0.8 μM olaparib (FIG. 15B); and 800 pM IMGN779, 0.8 μM niraparib, or 800 pm IMGN779+0.8 μM niraparib (FIG. 15C).

### DETAILED DESCRIPTION OF THE INVENTION

The present invention features methods of treating patients with cancers, e.g., a hematologic cancer, such as AML, by administering a combination of a CD33-targeted ADC containing an indolino-benzodiazepine dimer cytotoxic payload, in particular, the ADC of Formula (I) and a PARP inhibitor.

The invention is based, at least in part, on the discovery that the combination of IMGN779, a CD33-targeted antibody drug conjugate comprising and anti-huCD33 antibody, also known as huMy9-6 or Z4681A, conjugated to a novel DNA-alkylating agent, DGN462, via a cleavable disulfide linker and olaparib is more active in vitro against primary patient AML cells and in vivo against AML xenografts in mice than the individual agents alone.

### Anti-CD33 Antibodies

In one embodiment, the antibody in the ADC of formula (I), ADC1, or ADC2 is an anti-CD33 antibody, in particular, huMy9-6 antibody.

“My9-6”, “murine My9-6” and “muMy9-6”, is the murine anti-CD33 antibody from which huMy9-6 is derived. My9-6 is fully characterized with respect to the germline amino acid sequence of both light and heavy chain variable regions, amino acid sequences of both light and heavy chain variable regions, the identification of the CDRs, the identification of surface amino acids and means for its expression in recombinant form. See, for example, U.S. Pat. Nos. 7,557,189; 7,342,110; 8,119,787; 8,337,855 and U.S. Patent Publication No. 20120244171, each of which is incorporated herein by reference in their entirety. The amino acid sequences of muMy9-6 are also shown below in Table 1. The My9-6 antibody has also been functionally characterized and shown to bind with high affinity to CD33 on the surface of CD33-positive cells.

The term “variable region” is used herein to describe certain portions of antibody heavy chains and light chains that differ in sequence among antibodies and that cooperate in the binding and specificity of each particular antibody for its antigen. Variability is not usually evenly distributed throughout antibody variable regions. It is typically concentrated within three segments of a variable region called complementarity-determining regions (CDRs) or hypervariable regions, both in the light chain and the heavy chain variable regions. The more highly conserved portions of the variable regions are called the framework regions. The variable regions of heavy and light chains comprise four framework regions, largely adopting a beta-sheet configuration, with each framework region connected by the three CDRs, which form loops connecting the beta-sheet structure, and in some cases forming part of the beta-sheet structure. The CDRs in each chain are held in close proximity by the framework regions and, with the CDRs from the other chain, contribute to the formation of the antigen binding site of antibodies (E. A. Kabat et al. Sequences of Proteins of Immunological Interest, Fifth Edition, 1991, NIH). The “constant” region is not involved directly in binding an antibody to an antigen, but exhibits various effector functions, such as participation of the antibody in antibody-dependent cellular toxicity.

TABLE 1

| | Amino acid sequence |
|---|---|
| Heavy chain | SYYIH (SEQ ID NO: 1) |
| variable region | |
| complementary | |
| determining | |
| region CDR1 | |
| (murine and | |
| humanized) | |
| Heavy chain | VIYPGNDDISYNQKFXG, |
| variable region | (SEQ ID NO: 2) |
| complementary | wherein X is K or Q |
| determining | |
| region CDR2 | |
| (murine and | |
| humanized) | |
| Heavy chain | EVRLRYFDV (SEQ ID NO: 3) |
| variable region | |
| complementary | |
| determining | |
| region CDR3 | |
| (murine and | |
| humanized) | |
| Light chain | KSSQSVFFSSSQKNYLA |
| variable region | (SEQ ID NO: 4) |
| complementary | |
| determining | |
| region CDR1 | |
| (murine and | |
| humanized) | |
| Light chain | WASTRES (SEQ ID NO: 5) |
| variable region | |
| complementary | |
| determining | |
| region CDR2 | |
| (murine and | |
| humanized) | |
| Light chain | HQYLSSRT (SEQ ID NO: 6) |
| variable region | |
| complementary | |
| determining | |
| region CDR3 | |
| (murine and | |
| humanized) | |
| Murine heavy | QVQLQQPGAEVVKPGASVKMSCKASGYTFTSY |
| chain variable | YIHWIKQTPGQGLEWVGVIYPGNDDISYNQKF |
| region | KGKATLTADKSSTTAYMQLSSLTSEDSAVYYC |
| | AREVRLRYFDVWGAGTTVTVSS (SEQ ID |
| | NO: 7) |
| Murine light | NIMLTQSPSSLAVSAGEKVTMSCKSSQSVFFS |
| chain variable | SSQKNYLAWYQQIPGQSPKLLIYWASTRESGV |
| region | PDRFTGSGSGTDFTLTISSVQSEDLAIYYCHQ |
| | YLSSRTFGGGTKLEIKR (SEQ ID NO: 8) |

Humanized versions of My9-6, variously designated herein as “huMy9-6”, and “humanized My9-6”, are also described.

The goal of humanization is a reduction in the immunogenicity of a xenogenic antibody, such as a murine antibody, for introduction into a human, while maintaining the full antigen binding affinity and specificity of the antibody. Humanized antibodies may be produced using several technologies, such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host.

Strategies and methods for the resurfacing of antibodies, and other methods for reducing immunogenicity of antibodies within a different host, are disclosed in U.S. Pat. No. 5,639,641 (Pedersen et al.), which is hereby incorporated in its entirety by reference. Briefly, in a preferred method, (1) position alignments of a pool of antibody heavy and light chain variable regions are generated to give a set of heavy and light chain variable region framework surface exposed positions wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 angstroms of any atom of any residue of the complementarity-determining regions of the rodent antibody; and (5) the humanized rodent antibody having binding specificity is produced.

Antibodies can be humanized using a variety of other techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., 1991, Molecular Immunology 28(4/5):489-498; Studnicka G. M. et al., 1994, Protein Engineering 7(6):805-814; Roguska M. A. et al., 1994, PNAS 91:969-973), and chain shuffling (U.S. Pat. No. 5,565,332). Human antibodies can be made by a variety of methods known in the art including phage display methods. See also U.S. Pat. Nos. 4,444,887, 4,716,111, 5,545,806, and 5,814,318; and international patent application publication numbers WO 98/46645, WO 98/50433, WO 98/24893, WO 98/16654, WO 96/34096, WO 96/33735, and WO 91/10741 (said references incorporated by reference in their entireties).

As further described herein, the CDRs of My9-6 were identified by modeling and their molecular structures were predicted. Humanized My9-6 antibodies were then prepared and have been fully characterized as described, for example in U.S. Pat. Nos. 7,342,110 and 7,557,189, which are incorporated herein by reference. The amino acid sequences of the light and heavy chains of a number of huMy9-6 antibodies are described, for example, in U.S. Pat. Nos. 8,337,855 and 8,765,740, each of which is incorporated herein by reference. The amino acid sequences shown in Table 2 describe the huMy9-6 antibody of the invention.

TABLE 2

| | Amino acid sequence |
|---|---|
| Humanized | QVQLQQPGAEVVKPGASVKMSCKASGYTFTSYY |
| heavy chain | IHWIKQTPGQGLEWVGVIYPGNDDISYNQKFQG |
| variable | KATLTADKSSTTAYMQLSSLTSEDSAVYYCARE |
| region | VRLRYFDVWGQGTTVTVSS (SEQ ID NO: 9) |
| Humanized | EIVLTQSPGSLAVSPGERVTMSCKSSQSVFFSSS |
| light chain | QKNYLAWYQQIPGQSPRLLIYWASTRESGVPDRF |
| variable | TGSGSGTDFTLTISSVQPEDLAIYYCHQYLSSRT |
| region | FGQGTKLEIKR (SEQ ID NO: 10) |
| Full length | QVQLQQPGAEVVKPGASVKMSCKASGYTFTSYYI |
| humanized | HWIKQTPGQGLEWVGVIYPGNDDISYNQKFQGKA |
| heavy chain | TLTADKSSTTAYMQLSSLTSEDSAVYYCAREVRL |
| region | RYFDVWGQGTTVTVSSASTKGPSVFPLAPSSKST |
| | SGGTAALGCLVKDYFPEPVTVSWNSGALTSGVHT |
| | FPAVLQSSGLYSLSSVVTVPSSSLGTQTYICNVN |
| | HKPSNTKVDKKVEPKSCDKTHTCPPCPAPELLGG |
| | PSVFLFPPKPKDTLMISRTPEVTCVVVDVSHEDP |
| | EVKFNWYVDGVEVHNAKTKPREEQYNSTYRVVSV |
| | LTVLHQDWLNGKEYKCKVSNKALPAPIEKTISKA |
| | KGQPREPQVYTLPPSRDELTKNQVSLTCLVKGFY |
| | PSDIAVEWESNGQPENNYKTTPPVLDSDGSFFLY |
| | SKLTVDKSRWQQGNVFSCSVMHEALHNHYTQKSL |
| | SLSPG (SEQ ID NO: 11) |
| Full length | EIVLTQSPGSLAVSPGERVTMSCKSSQSVFFSSS |
| humanized | QKNYLAWYQQIPGQSPRLLIYWASTRESGVPDRF |
| light chain | TGSGSGTDFTLTISSVQPEDLAIYYCHQYLSSRT |
| region | FGQGTKLEIKRTVAAPSVFIFPPSDEQLKSGTAS |
| | VVCLLNNFYPREAKVQWKVDNALQSGNSQESVTE |
| | QDSKDSTYSLSSTLTLSKADYEKHKVYACEVTHQ |
| | GLSSPVTKSFNRGEC (SEQ ID NO: 12) |

Although epitope-binding fragments of the murine My9-6 antibody and the humanized My9-6 antibodies are discussed herein separately from the murine My9-6 antibody and the humanized versions thereof, it is understood that the term “antibody” or “antibodies” of the present invention may include both the full length muMy9-6 and huMy9-6 antibodies as well as epitope-binding fragments of these antibodies.

In a further embodiment, there are provided antibodies or epitope-binding fragments thereof comprising at least one complementarity-determining region having an amino acid sequence selected from the group consisting of SEQ ID NOs:1-6, and having the ability to bind CD33.

In a further embodiment, there are provided antibodies or epitope-binding fragments thereof comprising at least one heavy chain variable region and at least one light chain variable region, wherein said heavy chain variable region comprises three complementarity-determining regions having amino acid sequences represented by SEQ ID NOs:1-3, respectively, and wherein said light chain variable region comprises three complementarity-determining regions having amino acid sequences represented by SEQ ID NOs:4-6, respectively.

In a further embodiment, there are provided antibodies having a heavy chain variable region that has an amino acid sequence that shares at least 90% sequence identity with an amino acid sequence represented by SEQ ID NO:7, more preferably 95% sequence identity with SEQ ID NO:7, most preferably 100% sequence identity with SEQ ID NO:7.

Similarly, there are provided antibodies having a light chain variable region that has an amino acid sequence that shares at least 90% sequence identity with an amino acid sequence represented by SEQ ID NO:8, more preferably 95% sequence identity with SEQ ID NO:8, most preferably 100% sequence identity with SEQ ID NO:8.

In a further embodiment, antibodies are provided having a humanized (e.g., resurfaced, CDR-grafted) heavy chain variable region that shares at least 90% sequence identity with an amino acid sequence represented by SEQ ID NO:9, more preferably 95% sequence identity with SEQ ID NO:9, most preferably 100% sequence identity with SEQ ID NO:9.

Similarly, antibodies are provided having a humanized (e.g., resurfaced, CDR-grafted) light chain variable region that shares at least 90% sequence identity with an amino acid sequence corresponding to SEQ ID NO:10, more preferably 95% sequence identity with SEQ ID NO:10, most preferably 100% sequence identity with SEQ ID NO:10. In particular embodiments, the antibody includes conservative mutations in the framework region outside of the CDRs.

As used herein, “antibody fragments” include any portion of an antibody that retains the ability to bind to CD33, generally termed “epitope-binding fragments.” Examples of antibody fragments preferably include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Epitope-binding fragments, including single-chain antibodies, may comprise the variable region(s) alone or in combination with the entirety or a portion of the following: hinge region, C_{H1}, C_{H2}, and C_{H3} domains. Such fragments may contain one or both Fab fragments or the F(ab′)₂ fragment. Preferably, the antibody fragments contain all six CDRs of the whole antibody, although fragments containing fewer than all of such regions, such as three, four or five CDRs, are also functional. Further, the functional equivalents may be or may combine members of any one of the following immunoglobulin classes: IgG, IgM, IgA, IgD, or IgE, and the subclasses thereof. Fab and F(ab′)₂ fragments may be produced by proteolytic cleavage, using enzymes such as papain (Fab fragments) or pepsin (F(ab′)₂ fragments). The single-chain FVs (scFvs) fragments are epitope-binding fragments that contain at least one fragment of an antibody heavy chain variable region (V_{H}) linked to at least one fragment of an antibody light chain variable region (V_{L}). The linker may be a short, flexible peptide selected to assure that the proper three-dimensional folding of the (V_{L}) and (V_{H}) regions occurs once they are linked so as to maintain the target molecule binding-specificity of the whole antibody from which the single-chain antibody fragment is derived. The carboxyl terminus of the (V_{L}) or (V_{H}) sequence may be covalently linked by a linker to the amino acid terminus of a complementary (V_{L}) and (V_{H}) sequence. Single-chain antibody fragments may be generated by molecular cloning, antibody phage display library or similar techniques well known to the skilled artisan. These proteins may be produced, for example, in eukaryotic cells or prokaryotic cells, including bacteria.

The epitope-binding fragments of the present invention can also be generated using various phage display methods known in the art. In phage display methods, functional antibody domains are displayed on the surface of phage particles which carry the polynucleotide sequences encoding them. In particular, such phage can be utilized to display epitope-binding domains expressed from a repertoire or combinatorial antibody library (e.g., human or murine). Phage expressing an epitope-binding domain that binds the antigen of interest can be selected or identified with antigen, e.g., using labeled CD33 or CD33 bound or captured to a solid surface or bead. Phage used in these methods are typically filamentous phage including fd and M13 binding domains expressed from phage with Fab, Fv or disulfide-stabilized Fv antibody domains recombinantly fused to either the phage gene III or gene VIII protein.

Examples of phage display methods that can be used to make the epitope-binding fragments of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

After phage selection, the regions of the phage encoding the fragments can be isolated and used to generate the epitope-binding fragments through expression in a chosen host, including mammalian cells, insect cells, plant cells, yeast, and bacteria, using recombinant DNA technology, e.g., as described in detail below. For example, techniques to recombinantly produce Fab, Fab′ and F(ab′)₂ fragments can also be employed using methods known in the art such as those disclosed in PCT publication WO 92/22324; Mullinax et al., 1992, BioTechniques 12(6):864-869; Sawai et al., 1995, AJRI34:26-34; and Better et al., 1988, Science 240:1041-1043; said references incorporated by reference in their entireties. Examples of techniques which can be used to produce single-chain Fvs and antibodies include those described in U.S. Pat. Nos. 4,946,778 and 5,258,498; Huston et al., 1991, Methods in Enzymology 203:46-88; Shu et al., 1993, PNAS 90:7995-7999; Skerra et al., 1988, Science 240:1038-1040.

Also included within the scope of the invention are functional equivalents of the My9-6 antibody and the humanized My9-6 antibodies. The term “functional equivalents” includes antibodies with homologous sequences, chimeric antibodies, modified antibody and artificial antibodies, for example, wherein each functional equivalent is defined by its ability to bind to CD33. The skilled artisan will understand that there is an overlap in the group of molecules termed “antibody fragments” and the group termed “functional equivalents.”

Antibodies with homologous sequences are those antibodies with amino acid sequences that have sequence identity or homology with amino acid sequence of the murine My9-6 and humanized My9-6 antibodies of the present invention. Preferably identity is with the amino acid sequence of the variable regions of the murine My9-6 and humanized My9-6 antibodies of the present invention. “Sequence identity” and “sequence homology” as applied to an amino acid sequence herein is defined as a sequence with at least about 90%, 91%, 92%, 93%, or 94% sequence identity, and more preferably at least about 95%, 96%, 97%, 98%, or 99% sequence identity to another amino acid sequence, as determined, for example, by the FASTA search method in accordance with Pearson and Lipman, Proc. Natl. Acad. Sci. USA 85, 2444-2448 (1988).

As used herein, a chimeric antibody is one in which different portions of an antibody are derived from different animal species. For example, an antibody having a variable region derived from a murine monoclonal antibody paired with a human immunoglobulin constant region. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entireties.

The CDRs are of primary importance for epitope recognition and antibody binding. However, changes may be made to the residues that comprise the CDRs without interfering with the ability of the antibody to recognize and bind its cognate epitope. For example, changes that do not affect epitope recognition, yet increase the binding affinity of the antibody for the epitope may be made.

Thus, also included in the scope of the present invention are improved versions of both the murine and humanized antibodies, which also specifically recognize and bind CD33, preferably with increased affinity.

Several studies have surveyed the effects of introducing one or more amino acid changes at various positions in the sequence of an antibody, based on the knowledge of the primary antibody sequence and on its properties such as binding and level of expression (Yang, W. P. et al., 1995, J. Mol. Biol., 254, 392-403; Rader, C. et al., 1998, Proc. Natl. Acad. Sci. USA, 95, 8910-8915; Vaughan, T. J. et al., 1998, Nature Biotechnology, 16, 535-539).

In these studies, equivalents of the primary antibody have been generated by changing the sequences of the heavy and light chain genes in the CDR1, CDR2, CDR3, or framework regions, using methods such as oligonucleotide-mediated site-directed mutagenesis, cassette mutagenesis, error-prone PCR, DNA shuffling, or mutator-strains of E. coli (Vaughan, T. J. et al., 1998, Nature Biotechnology, 16, 535-539; Adey, N. B. et al., 1996, Chapter 16, pp. 277-291, in “Phage Display of Peptides and Proteins”, Eds. Kay, B. K. et al., Academic Press). These methods of changing the sequence of the primary antibody have resulted in improved affinities of the secondary antibodies (Gram, H. et al., 1992, Proc. Natl. Acad. Sci. USA, 89, 3576-3580; Boder, E. T. et al., 2000, Proc. Natl. Acad. Sci. USA, 97, 10701-10705; Davies, J. and Riechmann, L., 1996, Immunotechnolgy, 2, 169-179; Thompson, J. et al., 1996, J. Mol. Biol., 256, 77-88; Short, M. K. et al., 2002, J. Biol. Chem., 277, 16365-16370; Furukawa, K. et al., 2001, J. Biol. Chem., 276, 27622-27628).

By a similar directed strategy of changing one or more amino acid residues of the antibody, the antibody sequences described herein (e.g., in Tables 1 and 2) can be used to develop anti-CD33 antibodies with improved functions, including improved affinity for CD33. Improved antibodies also include those antibodies having improved characteristics that are prepared by the standard techniques of animal immunization, hybridoma formation and selection for antibodies with specific characteristics.

CD33-targeted Antibody Drug Conjugates

In certain embodiments, the present invention provides a method of treating a cancer, e.g., a hematologic cancer, in a subject comprising administering to the subject an effective amount of olaparib or a pharmaceutically acceptable salt thereof and an effective amount of an ADC of Formula (I):

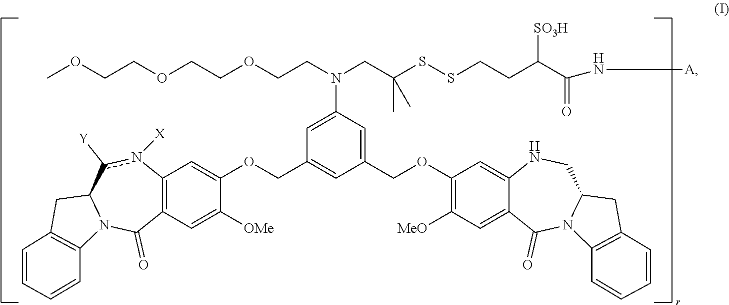

or a pharmaceutically acceptable salt thereof. The double line between N and C represents either a single bond or a double bond, provided that when it is a double bond, X is absent and Y is hydrogen; and when it is a single bond, X is hydrogen and Y is —SO₃H. The term “A” is the antibody or antigen-binding fragment thereof that specifically binds to CD33 comprising a heavy chain variable region (VH) complementary determining region (CDR)1 sequence of SEQ ID NO:1, a VH CDR2 sequence of SEQ ID NO:2, and a VH CDR3 sequence of SEQ ID NO:3, and a light chain variable region (VL) CDR1 sequence of SEQ ID NO:4, a VL CDR2 sequence of SEQ ID NO:5, and a VL CDR3 sequence of SEQ ID NO:6. The term “r” is an integer from 1 to 10.

In one embodiment, the antibody or antigen-binding fragment thereof comprises a heavy chain variable region comprising an amino acid sequence having at least 95% identity to the amino acid sequence of SEQ ID NO:7 or 9. In another embodiment, the antibody or antigen-binding fragment thereof comprises a light chain variable region comprising an amino acid sequence having at least 95% identity to the amino acid sequence of SEQ ID NO:8 or 10. In one embodiment, the antibody is huMy9-6. In another embodiment, the antibody is a CDR-grafted or resurfaced antibody.

ADC1, ADC2, IMGN779, and pharmaceutically acceptable salts thereof, are specific examples of ADCs that can be used in the disclosed methods of treatment.

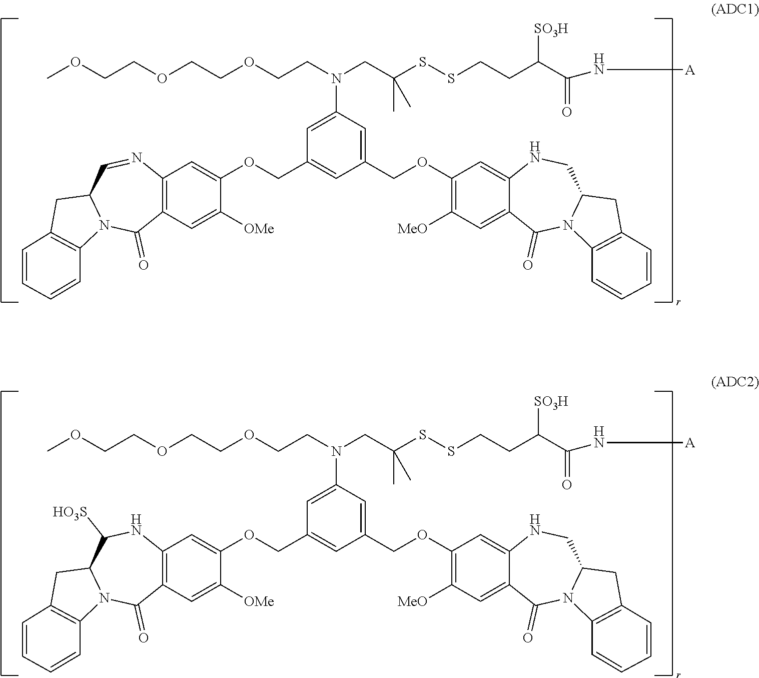

“A” is as defined for Formula (I). The term “r” is an integer from 1 to 10. Methods of preparing ADC1, ADC2, and IMGN779 are provided in U.S. Pat. Nos. 8,765,740 and 9,353,127, the entire teachings of which are incorporated herein by reference.

In other embodiments, the antibody portion of the ADC of formula (I), ADC1, or ADC2 is an anti-CD33 antibody comprising a heavy chain variable region having at least about 90%, 91%, 92%, 93%, or 94% sequence identity, and more preferably at least about 95%, 96%, 97%, 98%, or 99% sequence identity to SEQ ID NO:9 and a light chain variable region having at least about 90%, 91%, 92%, 93%, or 94% sequence identity, and more preferably at least about 95%, 96%, 97%, 98%, or 99% sequence identity to SEQ ID NO:10.

In particular embodiments, the antibody portion of the ADC of formula (I), ADC1 or ADC2 is the huMy9-6 antibody, also termed as “Z4681A.” In specific embodiments, the CD33-targeted ADC is IMGN779. IMGN779 comprises the huMy9-6 or Z4681A antibody, conjugated to DGN462, via a cleavable disulfide linker. IMGN779 may be represented as depicted below as ADC3:

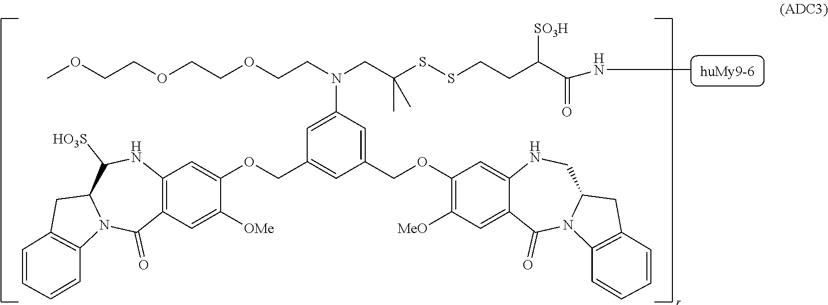

or a pharmaceutically acceptable salt thereof; or IMGN779 may also be represented below as ADC4:

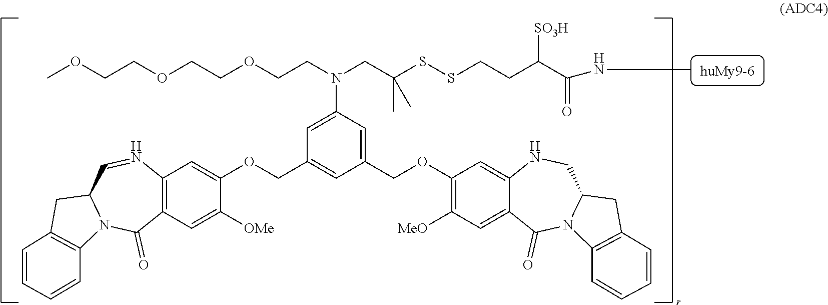

or a pharmaceutically acceptable salt thereof; or IMGN may also be a combination of ADC3 and ADC4.

In certain embodiments, the conjugate described herein may comprise 1-10 cytotoxic benzodiazepine dimer compounds, 2-9 cytototoxic benzodiazepine dimer compounds, 3-8 cytotoxic benzodiazepine dimer compounds, 4-7 cytotoxic benzodiazepine dimer compounds, or 5-6 cytotoxic benzodiazepine dimer compounds.

In certain embodiments, a composition comprising the conjugates described herein may comprise an average 1-10 cytotoxic benzodiazepine dimer molecule per antibody molecule. The average ratio of cytotoxic benzodiazepine dimer molecule per antibody molecule is referred to herein as the Drug Antibody Ratio (DAR). In one embodiment, the DAR is between 2-8, 3-7, 3-5 or 2.5-3.5.

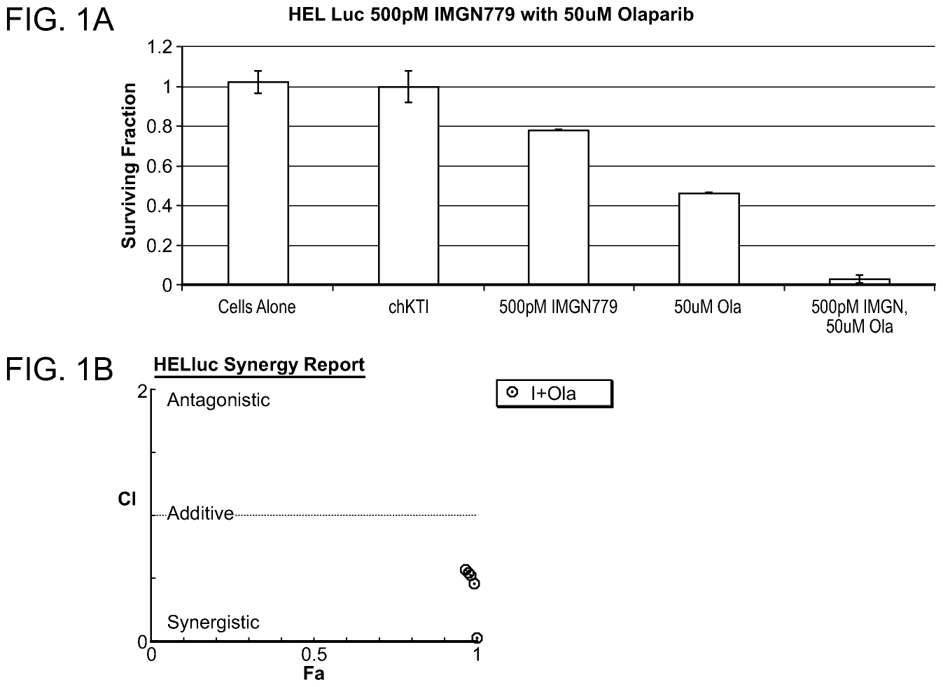

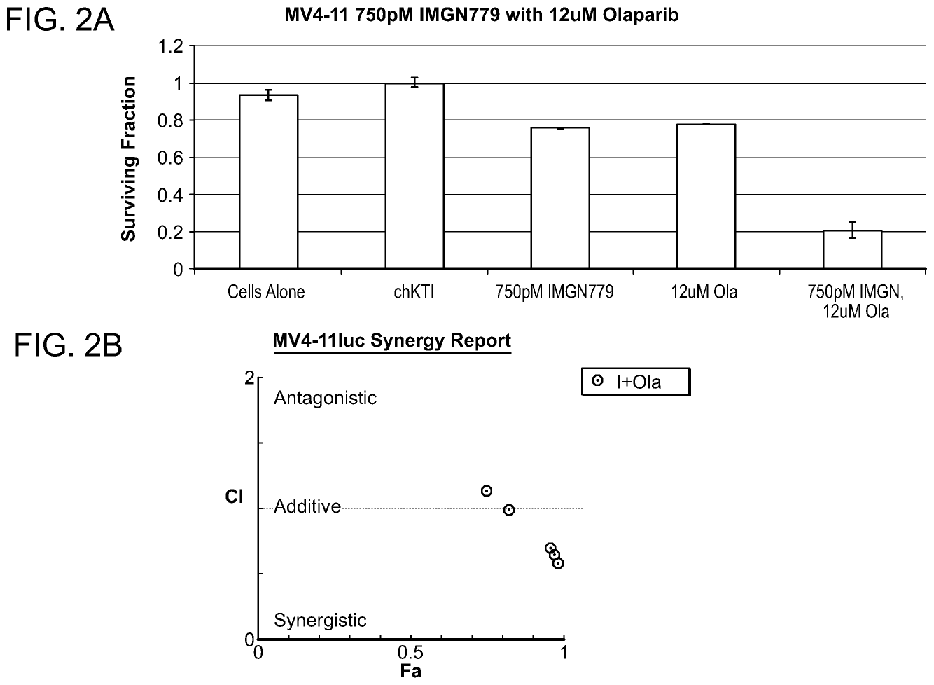

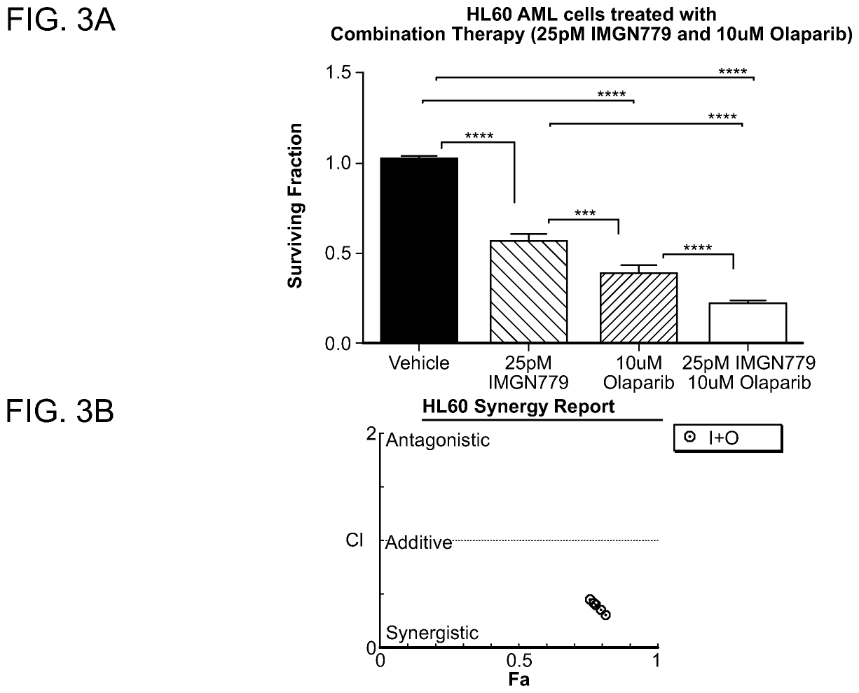

The cytotoxic benzodiazepine dimer compound and the conjugates described herein can be prepared according to methods described in U.S. Pat. Nos. 8,765,740 and 9,353,127, for example, but not limited to, paragraphs [0395]-[0397] and [0598]-[0607], FIGS. 1, 15, 22, 23, 38-41, 43, 48, 55 and 60, and Examples 1, 6, 12, 13, 20, 21, 22, 23, 26-30 and 32 of U.S. Pat. No. 8,765,740 and paragraphs [0007]-[0105], [0197]-[0291], FIGS. 1-11, 16, 28 and Examples 1-7, 9-13, 15 and 16 of U.S. Pat. No. 9,353,127.

The term “cation” refers to an ion with positive charge. The cation can be monovalent (e.g., Na⁺, K⁺, etc.), bi-valent (e.g., Ca²⁺, Me²⁺, etc.) or multi-valent (e.g., Al³⁺ etc.). Preferably, the cation is monovalent.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. In particular embodiments, the pharmaceutically acceptable salt is a sodium or a potassium salt.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

### PARP Inhibitors

PARP refers to a family of poly-ADP ribose polymerases that participate in a variety of DNA related functions including cell proliferation, differentiation, apoptosis, DNA repair and also has effects on telomere length and chromosome stability (d'Adda di Fagagna et al, 1999, Nature Gen., 23(1): 76-80). “PARP inhibitor” refers to substances that selectively bind to the poly-ADP ribose polymerase enzyme and decrease its activity. In one aspect, the PARP inhibitors used in the disclosed methods inhibit PARP-1 or PARP-2. PARP-1 is a poly-ADP ribose polymerase encoded by the PARP-1 gene. (See NCBI, 2016, PARP1 poly(ADP-ribose) polymerase 1, [Homo sapiens (human)]. PARP-2 is a poly-ADP ribose polymerase encoded by the PARP-1 gene. (See NCBI, 2016, PARP2 poly(ADP-ribose) polymerase 2, [Homo sapiens (human)]. PARP inhibitors can be identified using methods known in the art. See, for example, Cheung, et al. “A scintillation proximity assay for poly(ADP-ribose) polymerase,” Anal. Biochem. 2000, Vol. 282, pp. 24-28.

Suitable PARP inhibitors include those which are designed as analogs of benzamides, which bind competitively with the natural substrate NAD⁺ in the catalytic site of PARP. These PARP inhibitors include, but are not limited to, benzamides, quinolones and isoquinolones, benzopyrones, methyl 3,5-diiodo-4-(4′-methoxy-3′,5′-diiodo-phenoxy) benzoate (U.S. Pat. Nos. 5,464,871, 5,670,518, 5,922,775, 6,017,958, 5,736,576, and 5,484,951, each of which is incorporated herein by reference in its entirety). Other suitable PARP inhibitors include a variety of cyclic benzamide analogs (i.e. lactams) which are potent inhibitors at the NAD⁺ site. Other PARP inhibitors include, but are not limited to, benzimidazoles and indoles (see, e.g., EP 841924, EP 127052, U.S. Pat. Nos. 6,100,283, 6,310,082, US 2002/156050, US 2005/054631, WO 05/012305, WO 99/11628, and US 2002/028815, each of which is incorporated herein by reference in its entirety).

PARP inhibitors may possess the following structural characteristics: 1) amide or lactam functionality; 2) an NH proton of this amide or lactam functionality could be conserved for effective bonding; 3) an amide group attached to an aromatic ring or a lactam group fused to an aromatic ring; 4) optimal cis-configuration of the amide in the aromatic plane; and 5) constraining mono-aryl carboxamide into heteropolycyclic lactams (Costantino et al., 2001, J Med Chem., 44:3786-3794); Virag et al., 2002, Pharmacol Rev., 54:375-29, the latter of which summarizes various PARP inhibitors, and each of which is incorporated herein by reference in its entirety. Some of the examples of PARP inhibitors include, but are not limited to, isoquinolinone and dihydrolisoquinolinone (for example, U.S. Pat. No. 6,664,269, and WO 99/11624, each of which is incorporated herein by reference in its entirety), nicotinamide, 3-aminobenzamide, monoaryl amides and bi-, tri-, or tetracyclic lactams, phenanthridinones (Perkins et al., 2001, Cancer Res., 61:4175-4183, incorporated herein by reference in its entirety), 3,4-dihydro-5-methyl-isoquinolin-1(2H)-one and benzoxazole-4-carboxamide (Griffin et al., 1995, Anticancer Drug Des, 10:507-514; Griffin et al., 1998, J Med Chem, 41:5247-5256; and Griffin et al., 1996, Pharm Sci, 2:43-48, each of which is incorporated herein by reference in its entirety), dihydroisoquinolin-1(2H)-nones, 1,6-naphthyridine-5(6H)-ones, quinazolin-4(3H)-ones, thieno[3,4-c]pyridin-4(5H)ones and thieno[3,4-d]pyrimidin-4(3H)-ones, 1,5-dihydroxyisoquinoline, and 2-methyl-quinazolin-4[3H]-one (Yoshida et al., 1991, J Antibiot (Tokyo) 44: 111-112; Watson et al., 1998, Bioorg Med Chem., 6:721-734; and White et al., 2000, J Med Chem., 43:4084-4097, each of which is incorporated herein by reference in its entirety), 1,8-Napthalimide (Banasik et al., 1992, J Biol Chem, 267:1569-1575; Watson et al., 1998, Bioo 2001, Nat Med., 7: 108-113; Li et al., 2001, Bioorg Med Chem Lett., 11:1687-1690 30:1071-1082, each of which is incorporated herein by reference in its entirety), tetracyclic lactams, 1,11b-dihydro-[1]benzopyrano-[4,3,2-de]isoquinolin-3[2H]-one, 1-methyl-4-phenyl-1,2,3,6-tetrahydropyridine (MPTP) (Zhang et al., 2000, Biochem Biophys Res Commun., 278:590-598; and Mazzon et al., 2001, Eur J Pharmacol, 415:85-94, each of which is incorporated herein by reference in its entirety). Other examples of PARP inhibitors include, but are not limited to, those detailed in the patents: U.S. Pat. Nos. 5,719,151, 5,756,510, 6,015,827, 6,100,283, 6,156,739, 6,310,082, 6,316,455, 6,121,278, 6,201,020, 6,235,748, 6,306,889, 6,346,536, 6,380,193, 6,387,902, 6,395,749, 6,426,415, 6,514,983, 6,723,733, 6,448,271, 6,495,541, 6,548,494, 6,500,823, 6,664,269, 6,677,333, 6,903,098, 6,924,284, 6,989,388, 6,277,990, 6,476,048, and 6,531,464, each of which is incorporated herein by reference in its entirety. Additional examples of PARP inhibitors include, but are not limited to, those detailed in the patent application publications: US 2004198693 A1, US 2004034078A1, US 2004248879A1, US 2004249841A 2005080096A1, US 2005171101A1, US 2005054631A1, WO 05054201A1, WO 05054209A1, WO 05054210A1, WO 05058843A1, WO 06003146A1, WO 06003147A1, WO 06003148A1, WO 06003150A1, and WO 05097750A1, each of which is incorporated herein by reference in its entirety.

The following are specific examples of PARP inhibitors which can be used in the disclosed inventions:

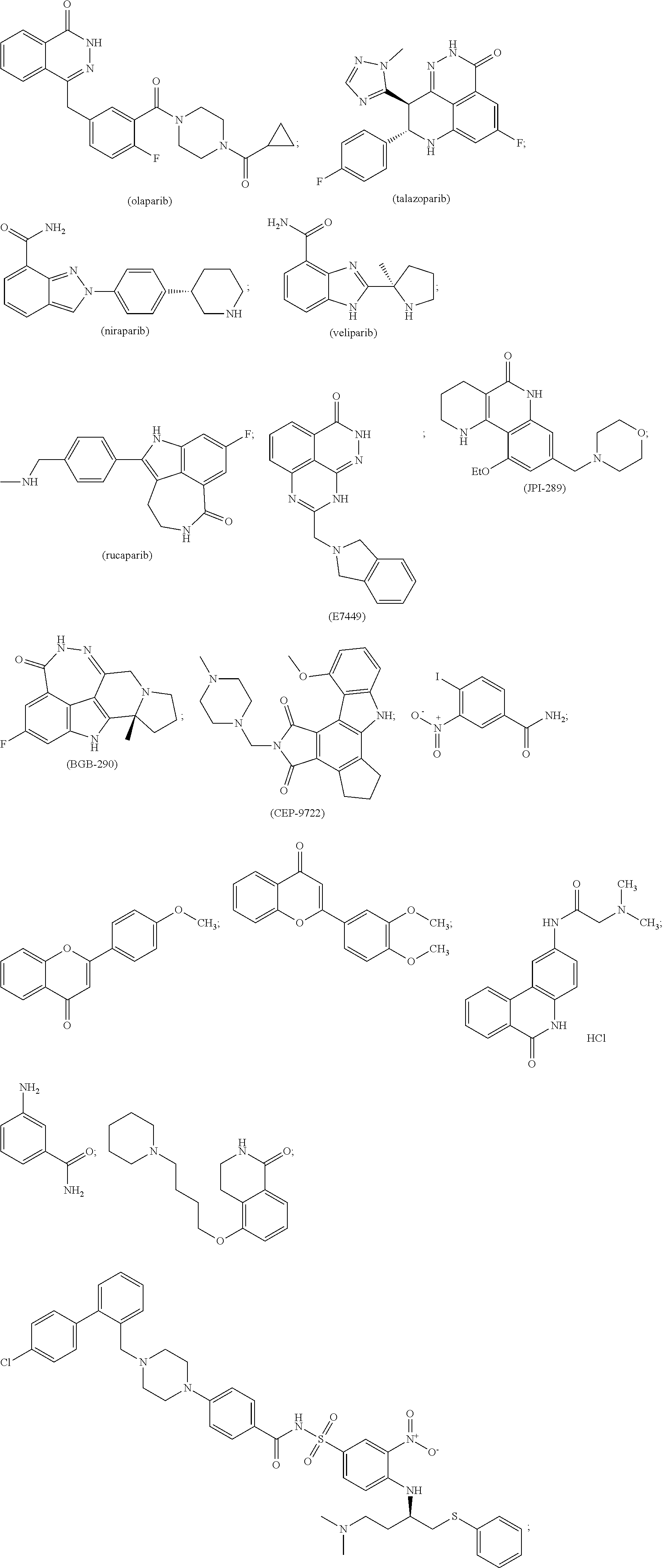

ABT767; and MP24, or a pharmaceutically acceptable salt thereof. In a particular embodiment, the present invention provides methods for treating cancer where the PARP inhibitor is olaparib, or a pharmaceutically acceptable salt thereof. In another particular embodiment, the present invention provides methods for treating cancer where the PARP inhibitor is talazoparib, or a pharmaceutically acceptable salt thereof.

### Therapeutic Applications

The present invention provides methods for treating patients with cancer, in particular a hematologic cancer, such as AML by administering a combination of a CD33-targeted ADC and a PARP inhibitor. As used herein, a “hematologic cancer” is a cancer that begins in blood-forming tissue, such as the bone marrow, or in the cells of the immune system. Examples of hematologic cancer are leukemia, lymphoma and multiple myeloma.

Cancers which can be treated using the disclosed methods include leukemia, lymphoma and myeloma. The cancer can be chemotherapy sensitive; alternatively, the cancer can be chemotherapy resistant. More specifically, cancers which can be treated using the disclosed methods include acute lymphoblastic leukemia (ALL), acute myeloid leukemia (AML), chronic lymphocytic leukemia (CLL), chronic myelogenous leukemia (CML), acute pro-myelocytic leukemia (APL), myelodysplastic syndromes (MDS), acute monocytic leukemia (AMOL), hairy cell leukemia (HCL), T-cell prolymphocytic leukemia (T-PLL), large granular lymphocytic leukemia, adult T-cell leukemia, small lymphocytic lymphoma (SLL), Hodgkin's lymphomas (Nodular sclerosis, Mixed cellularity, Lymphocyte-rich, Lymphocyte depleted or not depleted, and Nodular lymphocyte-predominant Hodgkin lymphoma), non-Hodgkin's lymphomas (all subtypes), chronic lymphocytic leukemia/Small lymphocytic lymphoma, B-cell prolymphocytic leukemia, lymphoplasmacytic lymphoma (such as Waldenström macroglobulinemia), splenic marginal zone lymphoma, plasma cell neoplasms (plasma cell myeloma, plasmacytoma, monoclonal immunoglobulin deposition diseases, heavy chain diseases), extranodal marginal zone B cell lymphoma (MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, Burkitt lymphoma/leukemia, T cell prolymphocytic leukemia, T cell large granular lymphocytic leukemia, Aggressive NK cell leukemia, Adult T cell leukemia/lymphoma, extranodal NK/T cell lymphoma (nasal type), enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma (unspecified), anaplastic large cell lymphoma), and multiple myeloma (plasma cell myeloma Kahler's disease).

In another embodiment, the cancer is selected from acute myeloid leukemia (AML), chronic myeloid leukemia (CML), acute lymphoblastic leukemia (ALL), B-cell lineage acute lymphoblastic leukemia (B ALL), chronic lymphocytic leukemia (CLL), hairy cell leukemia (HCL), myelodysplastic syndrome, basic plasmacytoid DC neoplasm (BPDCN) leukemia, non-Hodgkin lymphomas (NHL), mantle cell lymphoma, and Hodgkin's leukemia (HL). In another embodiment, the cancer is acute myeloid leukemia (AML). In yet another embodiment, the acute myeloid leukemia is refractory or relapse acute myeloid leukemia. In other embodiments, the invention provides treatment of patients with multi-drug resistant AML. P-glycoprotein (PGP), also known as MDR1, is an ATP-dependent drug efflux pump of 170 kD. It is a member of the ABC superfamily and is abundantly expressed in multidrug resistance (MDR) cells and produced by the ABCB1 gene. AML cells expressing PGP are, at least to some degree, resistant to treatment with conventional chemotherapeutics. Thus, the invention also provides methods for treating PGP-expressing AML.

The invention also provides methods of treating a hematologic cancer having at least one negative prognostic factor, e.g., overexpression of P-glycoprotein, overexpression of EVI1, a p53 alteration, DNMT3A mutation, FLT3 internal tandem duplication, and/or complex karyotype. In other embodiments, the invention also provides methods of treating a hematologic cancer having decreased expression in BRCA1, BRCA2, or PALB2 or mutations in BRCA1, BRCA2, or PALB2. Also within the scope of the invention is the selection of patients having at least one negative prognostic factor and/or decreased expression or mutations in BRCA1, BRCA2, or PALB2 prior to administration of the combination of a CD-33 targeted ADC and a PARP inhibitor.

In particular embodiments, the CD33-targeted ADC is administered to a subject in a pharmaceutically acceptable dosage form. ADCs may be administered intravenously as a bolus or by continuous infusion over a period of time, by intramuscular, subcutaneous, intra-articular, intrasynovial, intrathecal, oral, topical, or inhalation routes. Pharmaceutical compositions containing ADCs are administered by intratumoral, peritumoral, intralesional, or perilesional routes, to exert local as well as systemic therapeutic effects.

A pharmaceutically acceptable dosage form will generally include a pharmaceutically acceptable agent such as a carrier, diluent, and excipient. These agents are well known and the most appropriate agent can be determined by those of skill in the art as the clinical situation warrants. Examples of suitable carriers, diluents and/or excipients include: (1) Dulbecco's phosphate buffered saline, pH about 7.4, containing about 1 mg/ml to 25 mg/ml human serum albumin, (2) 0.9% saline (0.9% w/v NaCl), and (3) 5% (w/v) dextrose.

When present in an aqueous dosage form, rather than being lyophilized, the CD33-targeted ADC typically will be formulated at a concentration of about 0.1 mg/ml to 100 mg/ml, although wide variation outside of these ranges is permitted. For the treatment of disease, the appropriate dosage of the CD33-targeted ADC will depend on the type of disease to be treated, as defined above, the severity and course of the disease, the course of previous therapy, the patient's clinical history and response to the antibody, and the discretion of the attending physician. The antibody is suitably administered to the patient at one time or over a series of treatments.

In the disclosed methods, the ADC and the PARP inhibitor are administered in combination. A combination therapy is meant to encompass administration of the two or more therapeutic agents to a single subject, and are intended to include treatment regimens in which the agents are administered by the same or different route of administration or at the same or different times. These terms encompass administration of two or more agents to the subject so that both agents and/or their metabolites are present in the subject at the same time. They include simultaneous administration in separate compositions, simultaneous administration in the same composition and administration at different times in separate compositions.

The ADCs used in the disclosed methods and pharmaceutical compositions can be supplied as a solution or a lyophilized powder that are tested for sterility and for endotoxin levels. Suitable pharmaceutically acceptable carriers, diluents, and excipients are well known and can be determined by those of ordinary skill in the art as the clinical situation warrants.

Examples of suitable carriers, diluents and/or excipients include: (1) Dulbecco's phosphate buffered saline, pH about 7.4, containing or not containing about 1 mg/ml to 25 mg/ml human serum albumin, (2) 0.9% saline (0.9% w/v NaCl), and (3) 5% (w/v) dextrose; and may also contain an antioxidant such as tryptamine and a stabilizing agent such as Tween 20.

Pharmaceutical compositions are disclosed that include an ADC, a PARP inhibitor, and typically at least one additional substance, such as a pharmaceutically acceptable carrier or diluent. The pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. The Combination of IMGN779 and Olaparib Shows Enhanced Anti-Leukemic Activity, Reduces Cell Viability, Induces S-Phase Arrest, and Increases Cell Apoptosis In Vitro

Human CD33+ AML cell lines (HEL, MV4-11, and HL60) were treated in vitro with controls, IMGN779, olaparib, or IMGN779 and olaparib. Proliferation was measured by WST-8 reagent. Synergistic/additive effects were calculated using Compusyn software. Flow cytometry was performed to assess apoptosis, cell viability, and cell cycle effects.

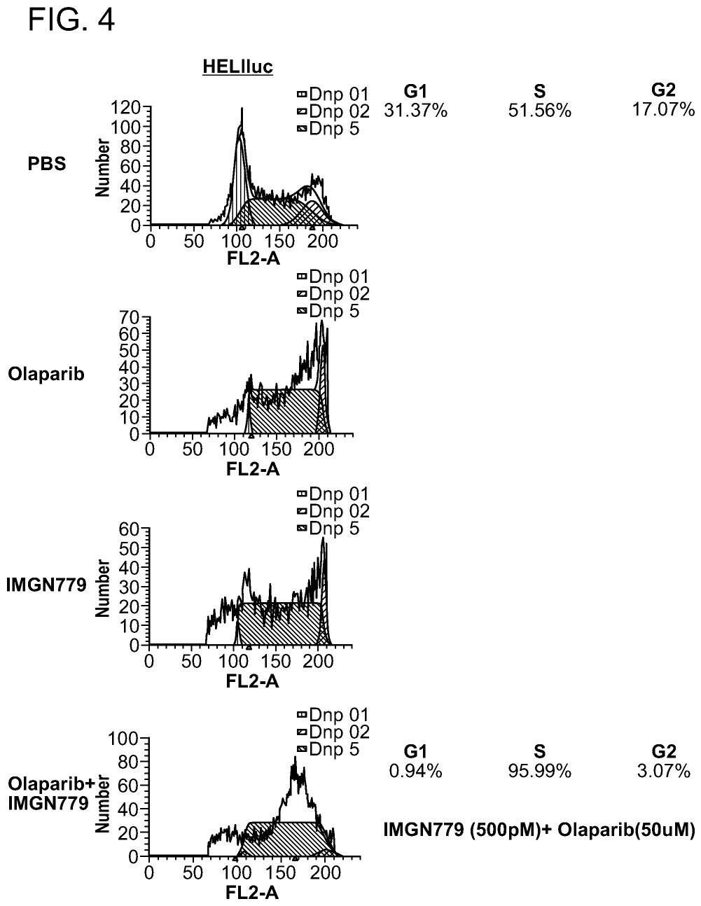

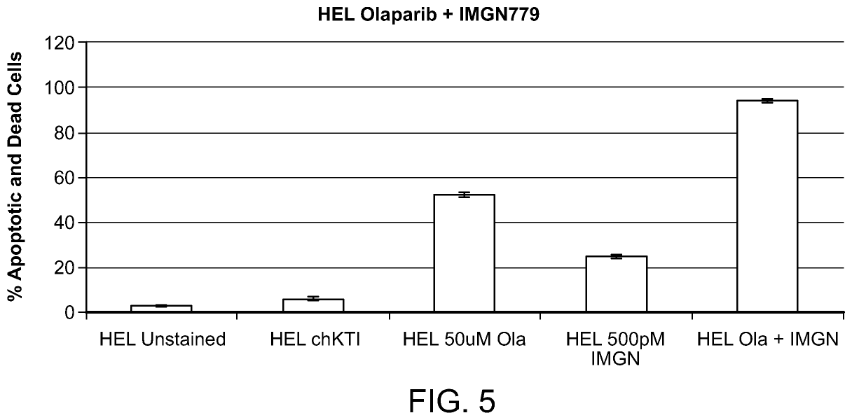

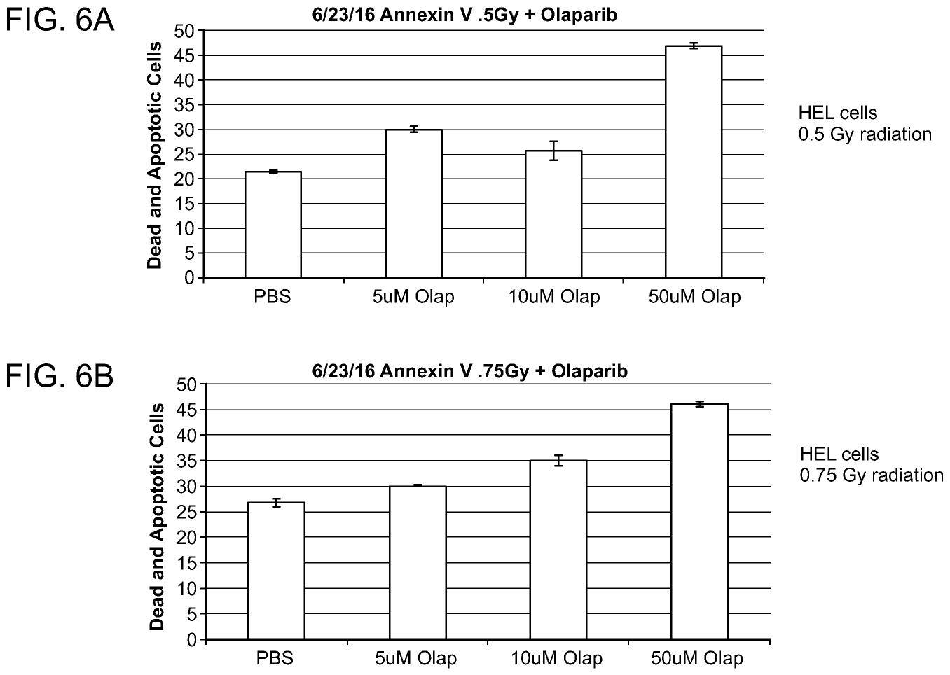

IMGN779 treatment induced significant growth inhibition in vitro in all CD33+ human AML cell lines tested that was dose dependent. The human AML cell lines tested highly expressed human CD33 and it was observed that IMGN779 cell killing was CD33 dependent. Olaparib also exerts dose-dependent grown inhibition in vitro in all CD33+ human AML cell lines tested and induced cell death in human AML cell lines via reversal of DNA damage repair mechanisms (data not shown). The combination treatment with IMGN779 (25 pM-750 pM) and olaparib (10-50 μM) significantly enhanced anti-leukemic effects over monotherapy in the same cell lines (FIGS. 1A, 2A, and 3A). Combination indices for IMGN779 and olaparib therapy ranged from 0.7-0.9, consistent with synergistic effects (FIGS. 1B, 2B, and 3B). The combination of IMGN779 and olaparib markedly reduced overall cell viability, increased apoptosis, and induced almost complete S-phase cell cycle arrest as compared with controls and single-agent treatments (FIG. 4 and FIG. 5). The effects on cell death after DNA-damaging radiation exposure in the presence of olaparib alone was also assessed showing a correlation in increase cell death and apoptosis with the increase in concentration of olaparib supporting the mechanism of action of PARP inhibitors (FIGS. 6A and 6B).

### Example 2. The Combination of IMGN779 and Olaparib Reduces AML Burden and Prolongs Survival in a Systemic AML Xenograft Model

In these experiments, stably transfected luciferase-positive human AML cells (HEL) characterized by high hCD33 expression levels were injected via tail vein into sublethally irradiated 6-8 week old SCID mice. Leukemia-engrafted animals were divided into treatment groups consisting of vehicle control, IMGN779 (15 mcg/kg), olaparib (100 mg/kg), or IMGN779+ olaparib (same doses). In vivo systemic leukemia disease burden was evaluated weekly by small animal bioluminescent imaging. Toxicity was determined by clinical examination and weight measurements throughout the experiment. Overall study endpoints were (a) time to overall morbidity/mortality and/or (b) change in leukemia disease burden as compared with vehicle-treated mice or mice treated with single agent therapy. 8-10 mice were used in control and experimental groups, as this has been found to be the minimum number of animals needed to determine statistical significance using the Cox-Mandel test. Simple stastical analyses will be performed utilizing the Stat Prism statistical software.

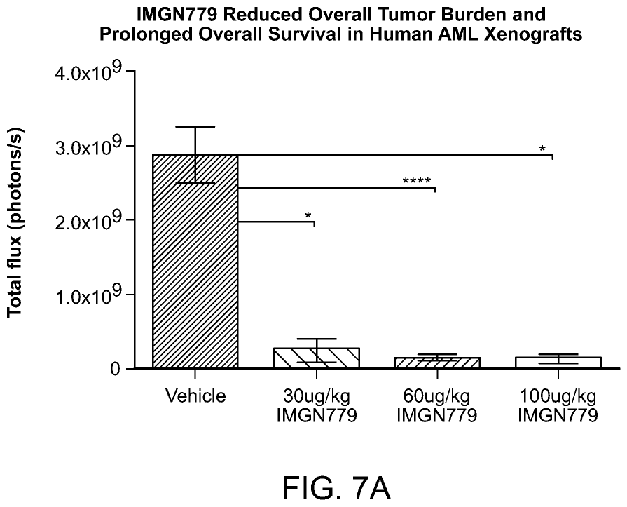

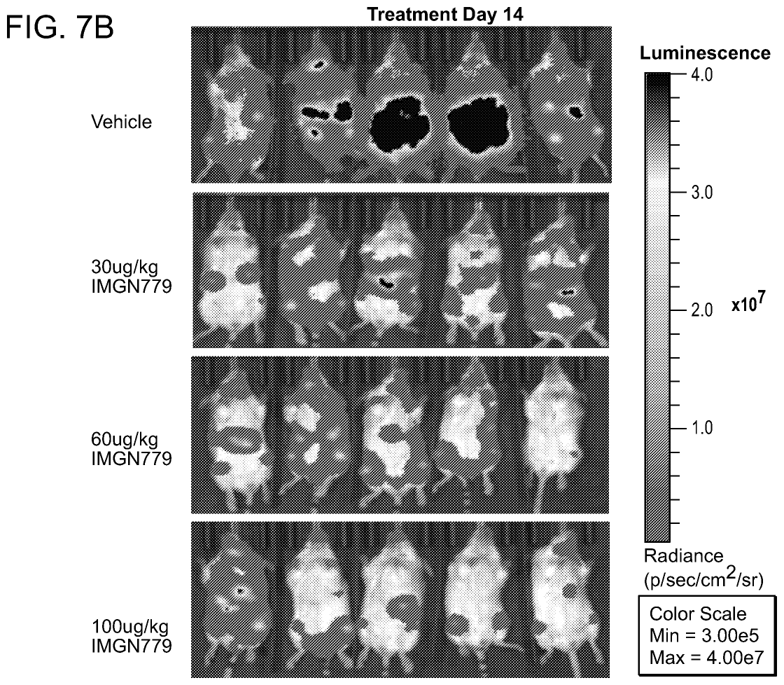

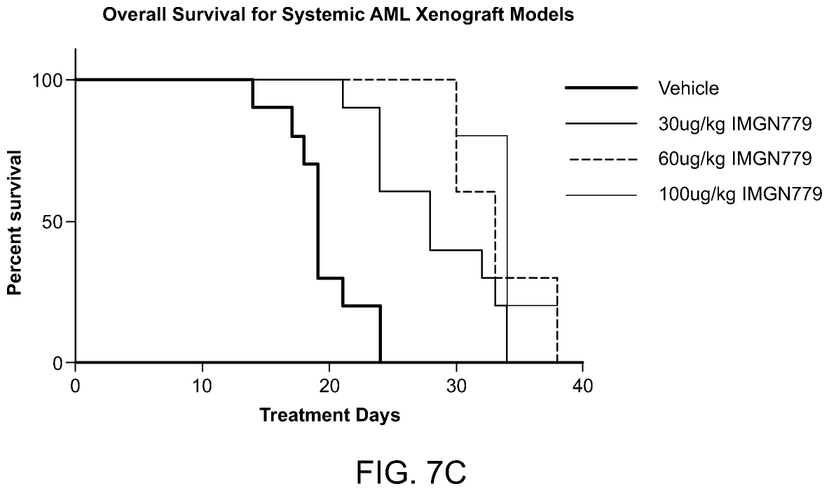

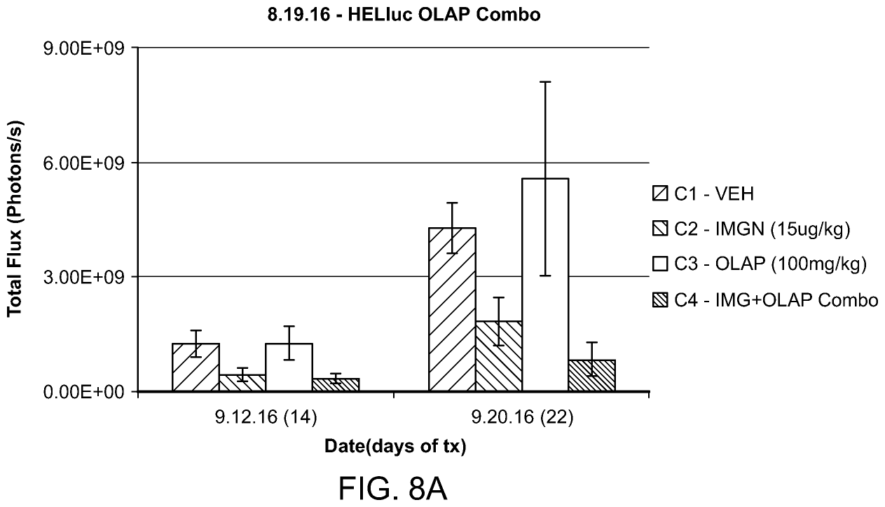

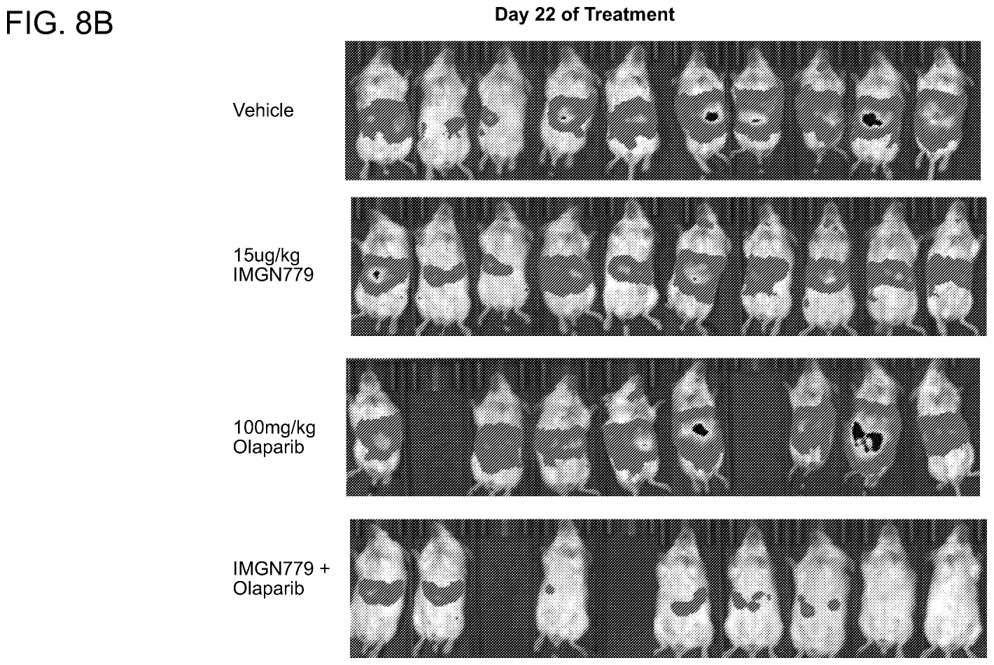

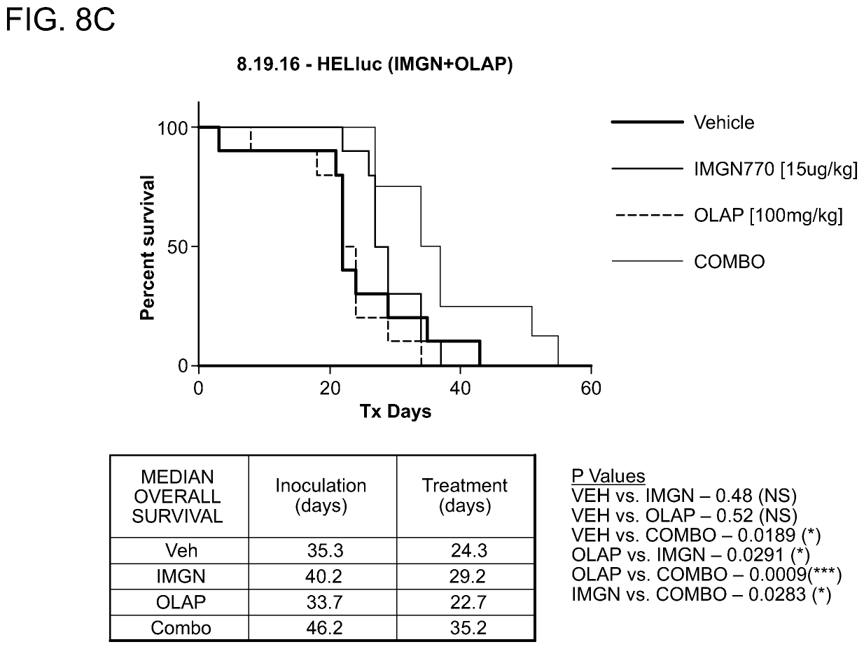

IMGN779 administered as a single dose ranging from 30 μg/kg to 100 μg/kg, by payload (0.5 mg/kg to 5 mg/kg, by antibody), was overall well tolerated in SCID mice bearing systemic human CD33+ AML (HEL-luciferase) xenografts. Significant dose-dependent anti-leukemic activity, as reflected by decreased leukemia burden and prolonged overall survival was observed (FIGS. 7A-7C). As shown in FIGS. 8A-8C, mice engrafted with human AML cells (HEL-luciferase) and treated with combination IMGN779 and olaparib therapy had significantly prolonged overall survival (median 46.2 days from inoculation) as compared with vehicle-treated (median 35.3 days, p 0.0189), IMGN779 alone (median 40.2 days, p 0.0283) or olaparib alone (median 33.7 days, p 0.0009) therapy. On treatment day 22, there was a significant decrease in overall leukemia disease burden as determined by total body bioluminescent flux in the combination treatment group mice as opposed to vehicle or monotherapy. The results of these studies demonstrate that the combination of IMGN779 and olaparib enhances anti-tumor activity in the HEL-luciferase systemic AML xenograft.

### Example 3. The Combination of IMGN779 and Olaparib Enhances Inhibition of Primary AML Colony Formation

The in vitro efficacy of IMGN779 on up to 50 clinically annotated patient AML samples with known CD33 expression levels, disease status (de novo vs. secondary vs. refractory/relapsed), karyotype, molecular aberrations (i.e. FLT-3 and NPM-1 mutation status), and response to therapy (if any) was evaluated.

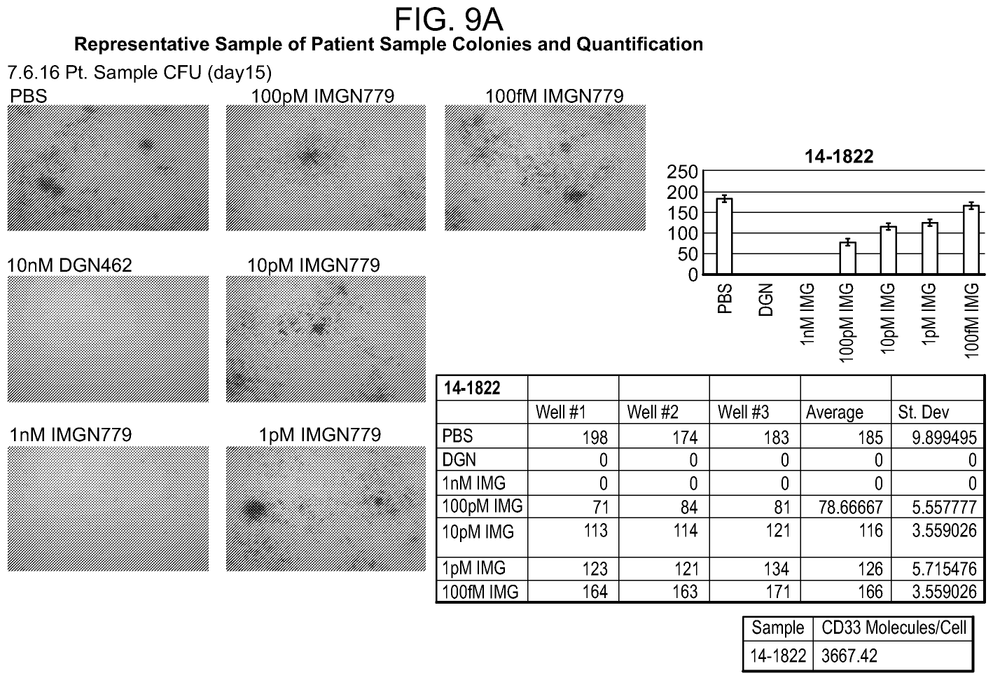

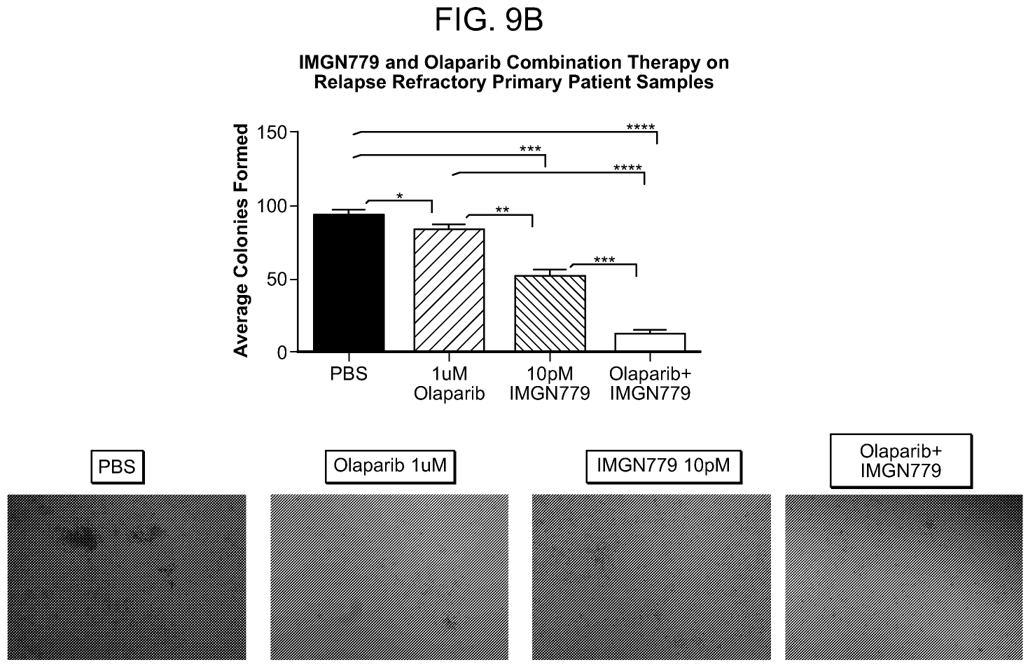

Short-term colony forming unit (CFU) assays were performed using cells obtained from multiple patients to evaluate the preclinical efficacy of IMGN779 in primary AML samples. Cryopreserved AML patient samples were obtained under IRB-approved protocols from the Roswell Park Hematologic Procurement Shared Resource. Cells were thawed on the day of the assay. Quantitation of surface expression of human CD33 molecules was performed on patient AML samples using Quantibright bead analysis on the same day. Thawed cells were evaluated for overall viability; samples with >50% viable cells were further quantified and exposed to vehicle (PBS) or varying concentrations of IMGN779 and/or olaparib in vitro for 24 hours prior to plating in semisolid methylcellulose medium for 13-15 days. CFU assays were quantified 13-15 days after methocult plating using a Spot-RT3 camera mounted to an inverted microscope with SPOT-Basic imaging software. A representative sample for each condition was captured and triplicate wells were averaged and reported (+/− standard deviation) as seen in FIG. 9A.

CFU assays with primary AML samples treated with vehicle and various concentrations of single agent IMGN779 CFUs were set up. Information on the clinical characteristics (specifically diagnostic cytogenetics and FLT-3 mutation status) was provided and was supplied by the RPCI Hematologic Procurement Shared Resource Facility under an IRB-approved protocol. It was found that IMGN779 inhibits primary AML sample colony formation in a dose dependent manner in a total of 15 primary AML samples. IMGN779 was combined with olaparib to verify the synergistic nature of the combination in primary AML samples. Combination doses were performed in triplicate. Representative sample images for each treatment condition are shown in FIG. 9B. An unpaired T test was used to determine significance among treatment groups. Exposure to a combination of IMGN779 and olaparib significantly inhibited CFU growth of progenitor cells established from bone marrow samples of patients (n=7) with relapsed/refractory, FLT3-ITD, and/or complex karyotype AML. As seen in FIG. 9B, statistically significant inhibition of viable CFUs was observed following combination IMGN779 (10 pM) and olaparib (1 μM) therapy as compared with monotherapy or vehicle controls (p<0.001). These results demonstrate that the combination of IMGN779 and olaparib can be effective in clinically chemo-resistant disease settings.

### Example 4. The Combination of IMGN779 and Niraparib and IMGN779 and Talazoparib Show Enhanced Anti-Leukemic Activity, Induce S-Phase Arrest, and Increase Cell Apoptosis and DNA Damage In Vitro

Human CD33+ AML cell lines (HEL-luc and HL60) were treated in vitro with IMGN779 at variable dose ranges (100 pM-1 nM) alone and in combination with each of the following PARP inhibitors: rucaparib, veliparib, talazoparib, and niraparib. Proliferation was measured following incubation with WST-8 reagent. Synergistic/additive effects were calculated using Compusyn software at varying drug concentrations. Flow cytometry for apoptosis, viability, DNA damage/repair, and cell cycle effects following combination versus single drug treatment were also performed.

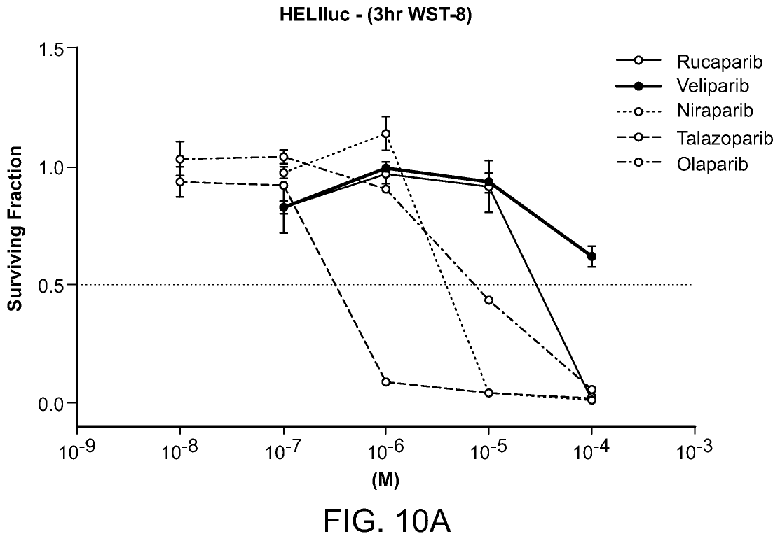

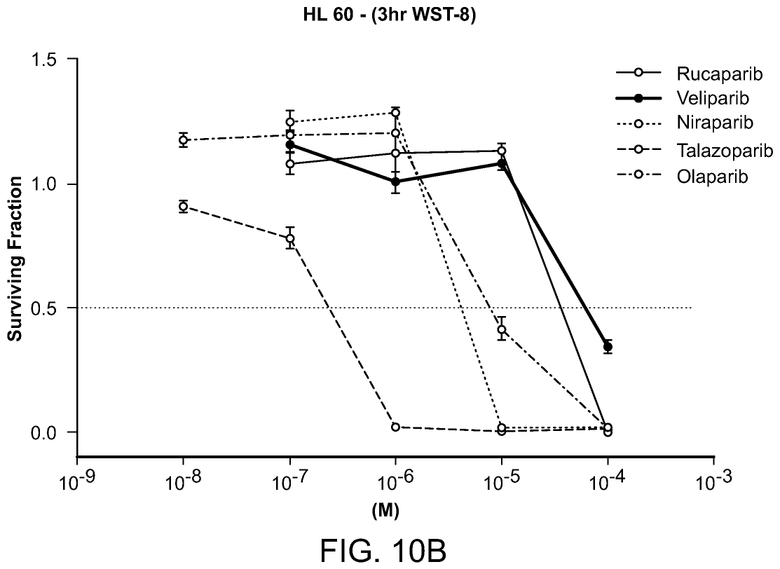

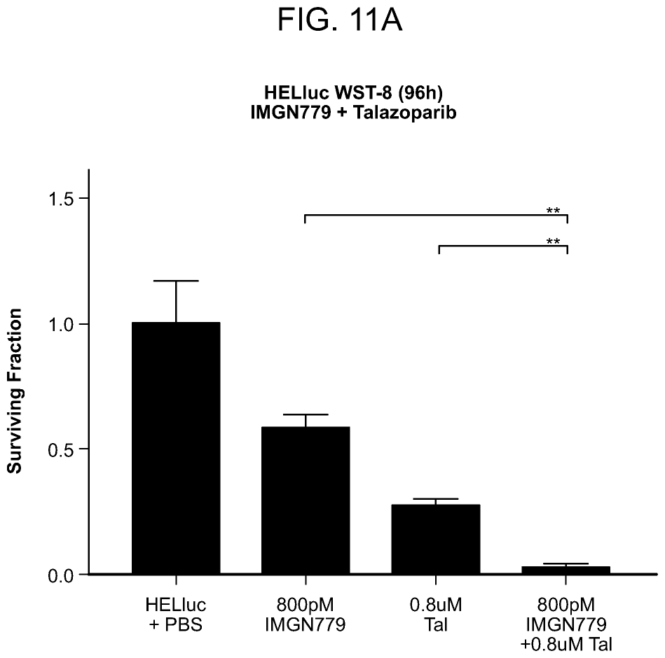

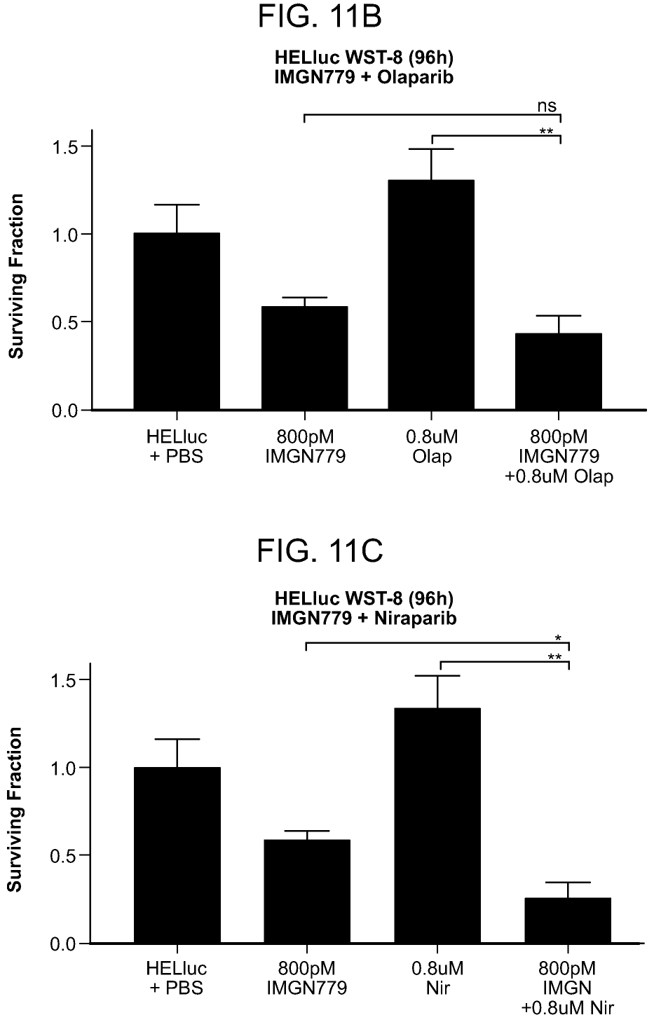

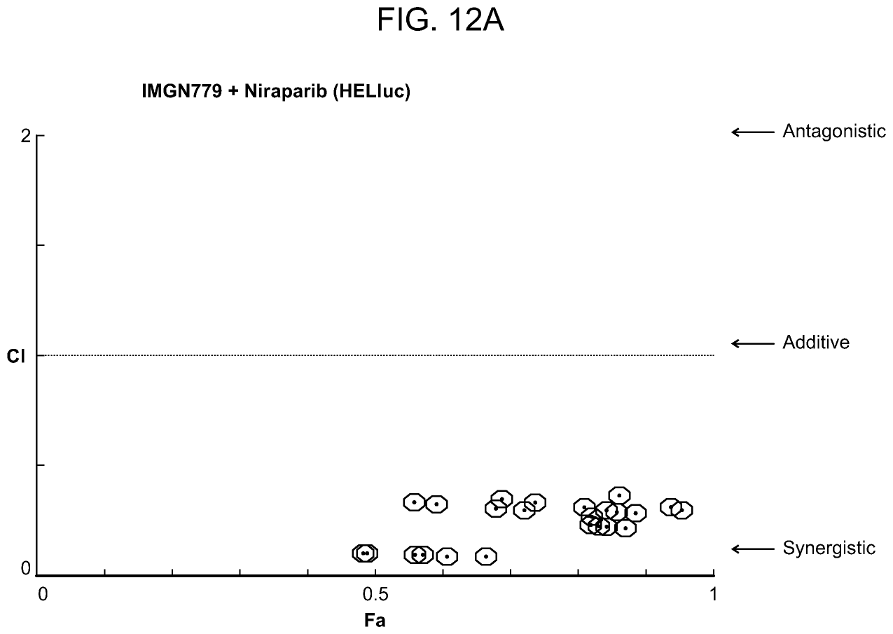

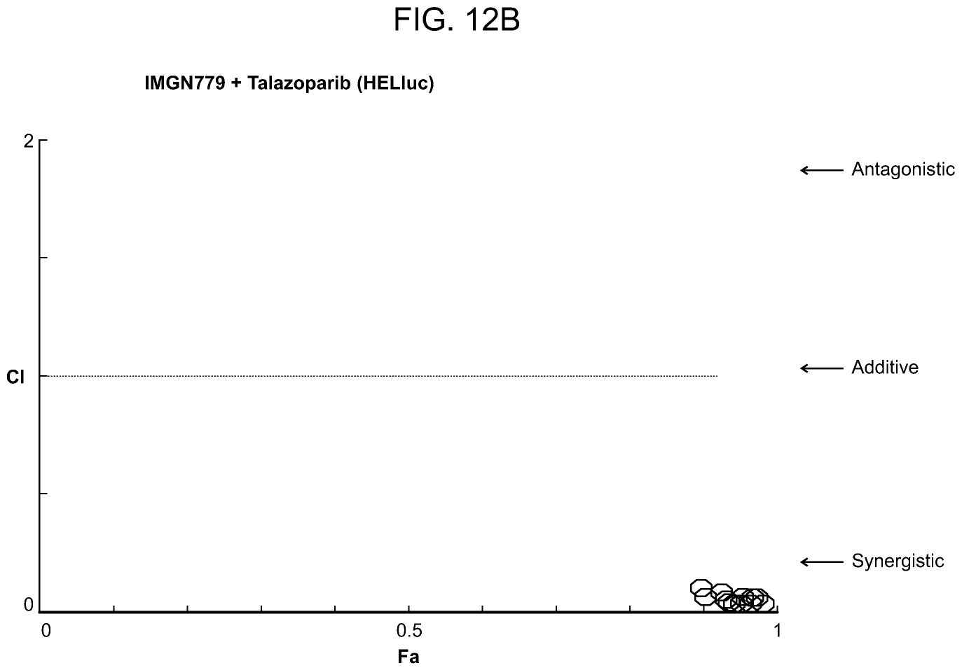

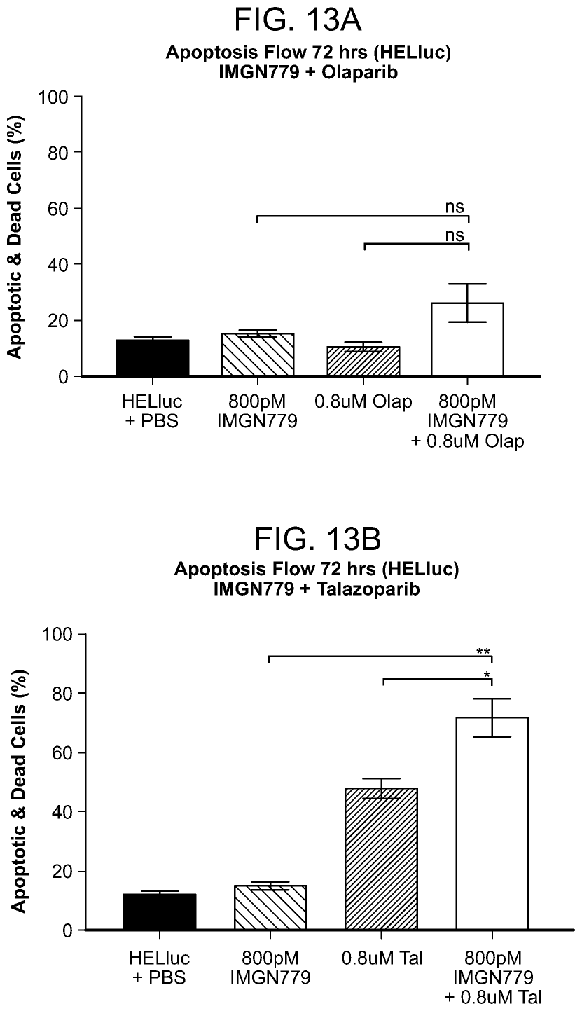

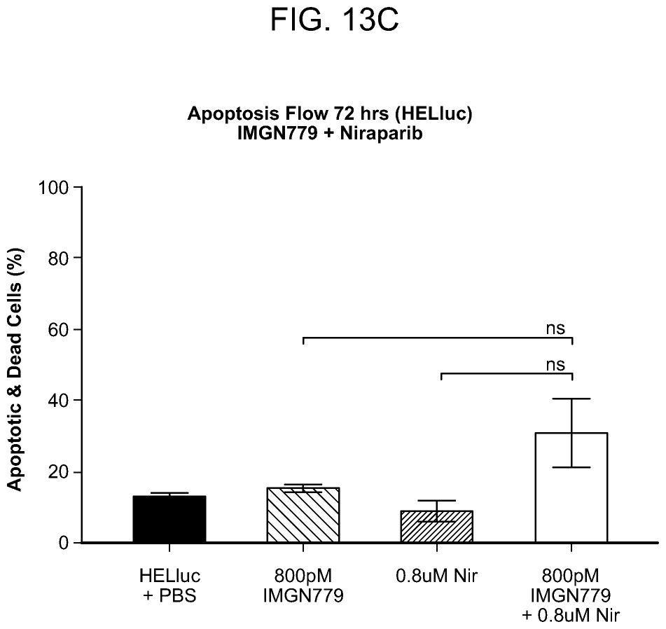

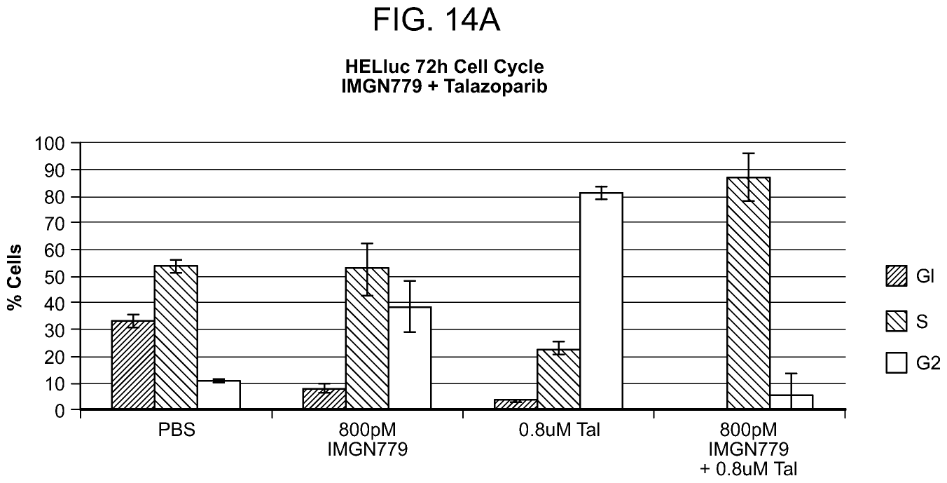

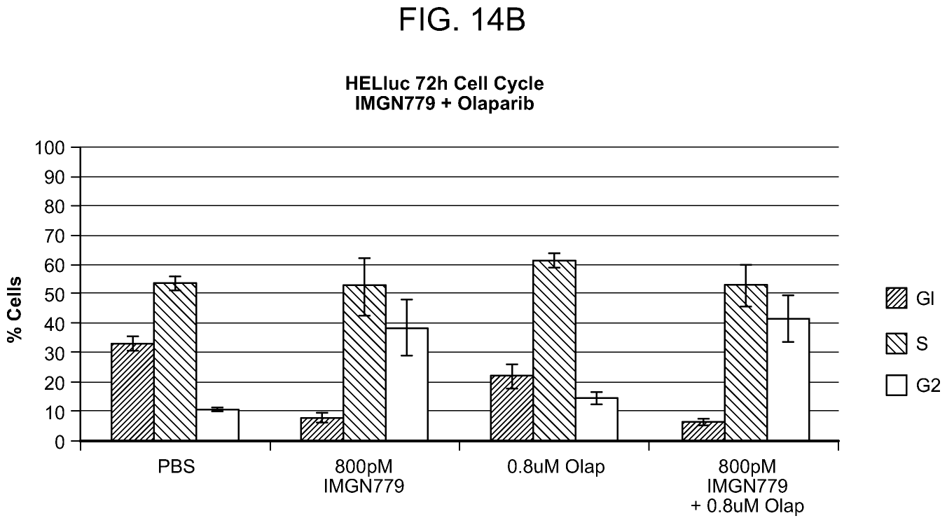

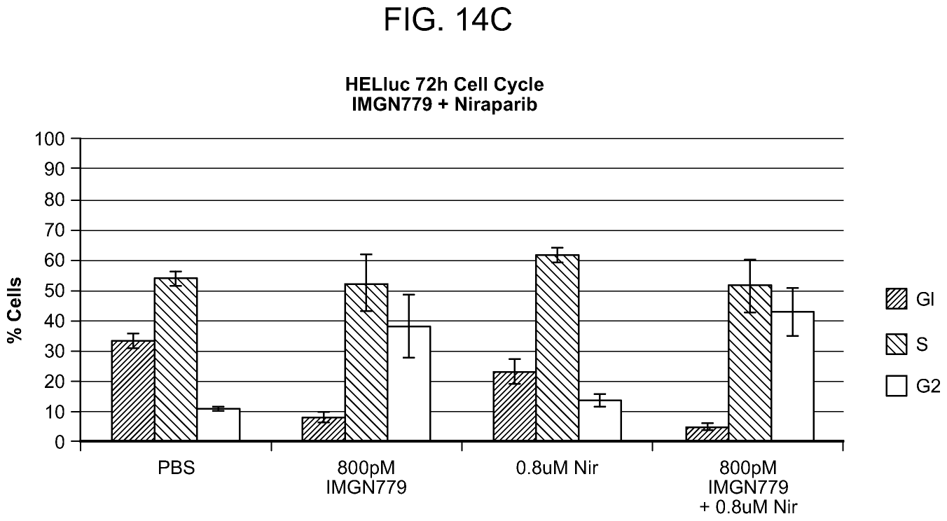

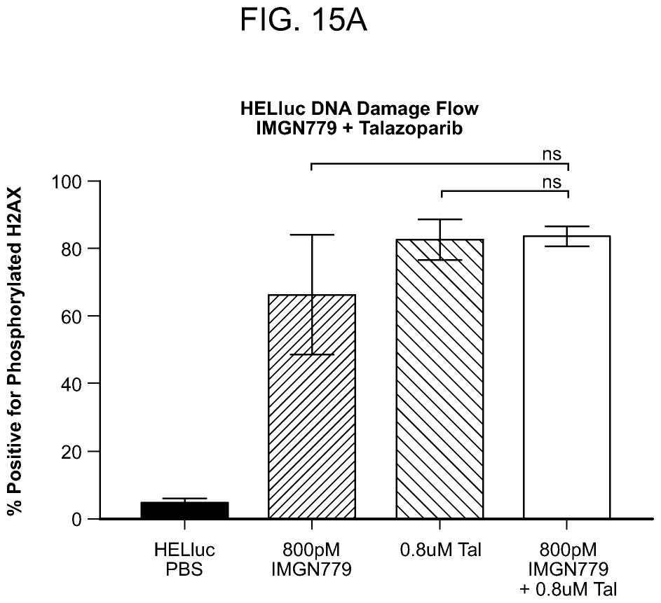

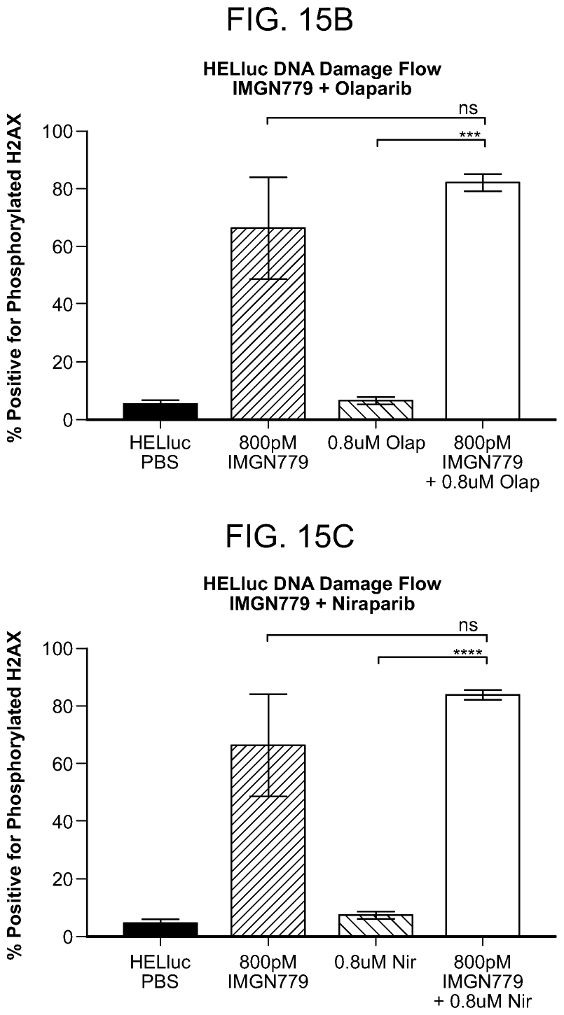

Treatment of rucaparib, veliparib, niraparib, talazoparib, and olaparib in HEL-luc and HL60 cell lines showed talazoparib to be the most potent PARP inhibitor in the cell lines tested (FIGS. 10A-10B, Table 3). The combination treatment with talazoparib (0.8 μM) alone and in combination with IMGN779 (800 pM) resulted in the greatest decrease in surviving fraction of cells in the same cell line as compared to combination treatment with olaparib and niraparib at the same concentrations (FIGS. 11A-11C). Combination indices for IMGN779+ talazoparib and IMGN779+ niraparib therapy as calculated by Compusyn are less than 1, consistent with synergistic effects (FIGS. 12A-12B). Further, the combination of IMGN779+ talazoparib, at the concentrations tested, resulted in the greatest increase in apoptosis (FIGS. 13A-13C), S-phase cell cycle arrest (FIGS. 14A-14C), and DNA damage (FIGS. 15A-15C), as compared to the combination of IMGN779+ niraparib and IMGN779+ olaparib in HEL-luc cell lines. The results of these experiments further support the mechanism of action of PARP inhibitors and supporting the use of PARP inhibitors in combination with IMGN779 for the treatment of cancer.

TABLE 3
IC50 values for PARP inhibitors in AML cell lines

| | Vellparib | Rucaparib | Olaparib | Niraparib | Talazoparib |
|---|---|---|---|---|---|
| | (μM) | (μM) | (μM) | (μM) | (μM) |
| HELluc | >100 | 39 | 7.4 | 7.3 | 0.4 |
| HL60 | 88 | 75 | 8.9 | 2.5 | 0.2 |

1. A method of treating cancer in a subject comprising the steps of administering to the subject an effective amount of a poly-ADP ribose polymerase (PARP) inhibitor and an effective amount of an antibody-drug conjugate of Formula (I) 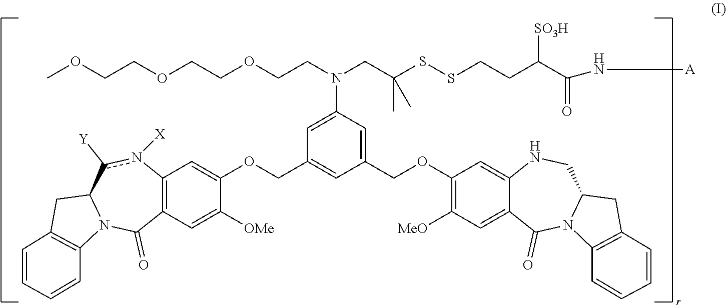
or a pharmaceutically acceptable salt thereof, wherein:
the double line [symbol] between N and C represents a single bond or a double bond, provided that when it is a double bond, X is absent and Y is hydrogen, when it is a single bond, X is hydrogen and Y is —SO₃H;
wherein A is the antibody or antigen-binding fragment thereof specifically binds to CD33 comprising a heavy chain variable region (VH) complementary determining region (CDR)1 sequence of SEQ ID NO:1, a VH CDR2 sequence of SEQ ID NO:2, and a VH CDR3 sequence of SEQ ID NO:3, and a light chain variable region (VL) CDR1 sequence of SEQ ID NO:4, a VL CDR2 sequence of SEQ ID NO:5, and a VL CDR3 sequence of SEQ ID NO:6; and
r is an integer from 1 to 10.

2. (canceled)

3. (canceled)

4. The method of claim 1, wherein the pharmaceutically acceptable salt is a sodium or potassium salt.

5. The method of claim 1, wherein the PARP inhibitor is a PARP-1 inhibitor or a PARP-2 inhibitor.

6. (canceled)

7. The method of claim 1, wherein the PARP inhibitor is selected from the group consisting of 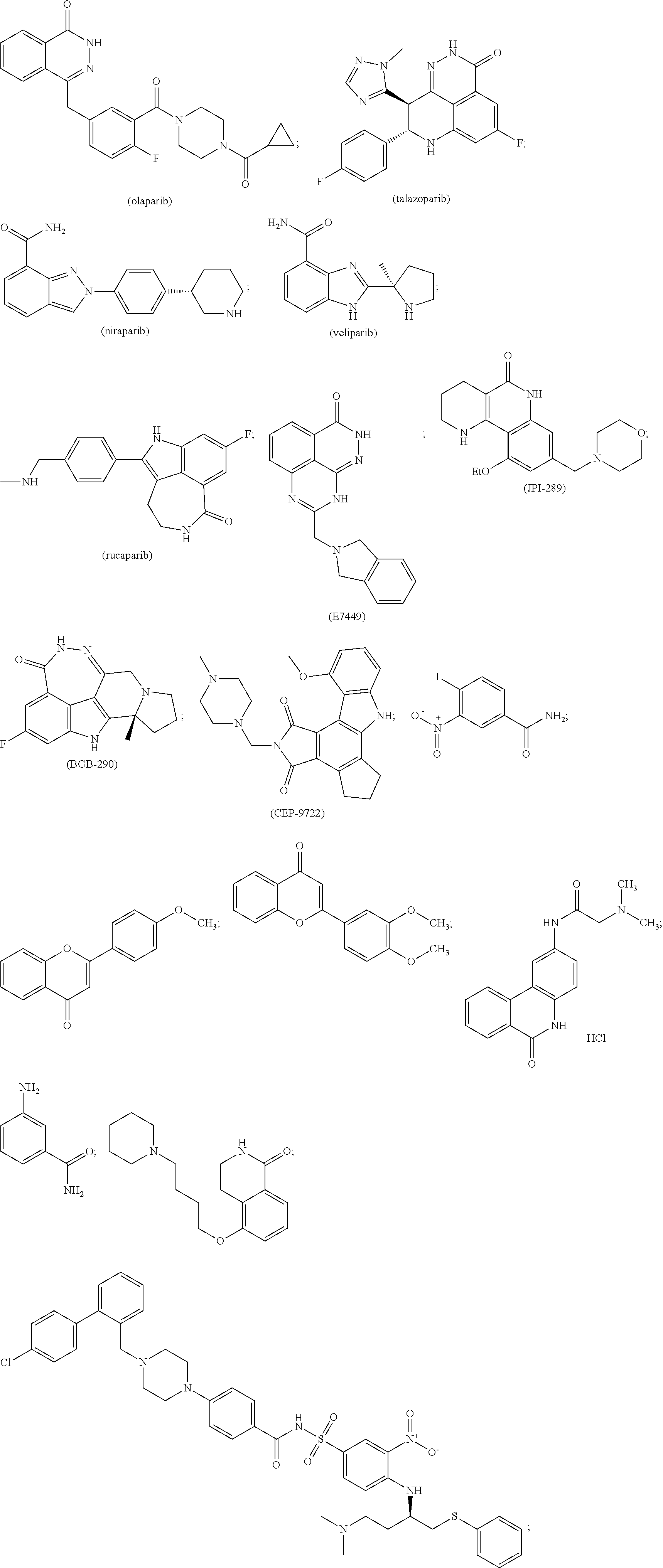
ABT767; and MP-124, or a pharmaceutically acceptable salt thereof.

8. (canceled)

9. The method of claim 1, wherein the antibody or antigen-binding fragment thereof comprises a heavy chain variable region comprising an amino acid sequence having at least 95% identity to the amino acid sequence of SEQ ID NO:7 or 9; and a light chain variable region comprising an amino acid sequence having at least 95% identity to the amino acid sequence of SEQ ID NO:8 or 10.

10. (canceled)

11. The method of claim 1, wherein the antibody or antigen-binding fragment thereof comprises a heavy chain variable region comprising the sequence of SEQ ID NO:9 and a light chain variable region comprising the sequence of SEQ ID NO:10.

12. The method of claim 1, wherein the antibody is huMy9-6.

13. The method of claim 12, wherein the antibody is a CDR-grafted or resurfaced antibody.

14. (canceled)

15. The method of claim 1, wherein the cancer is selected from the group consisting of leukemia, lymphoma and myeloma.

16. The method of claim 15, wherein the cancer is selected from the group consisting of acute myeloid leukemia (AML), chronic myeloid leukemia (CML), acute lymphoblastic leukemia (ALL), B-cell lineage acute lymphoblastic leukemia (B-ALL), chronic lymphocytic leukemia (CLL), hairy cell leukemia (HCL), myelodysplastic syndrome (MDS), basic plasmacytoid DC neoplasm (BPDCN) leukemia, non-Hodgkin lymphomas (NHL), mantle cell lymphoma, and Hodgkin's leukemia (HL).

17. The method of claim 16, wherein the cancer is acute myeloid leukemia (AML).

18. The method of claim 17, wherein the acute myeloid leukemia (AML) is refractory or relapse acute myeloid leukemia.

19. The method of claim 17, wherein the acute myeloid leukemia (AML) is characterized by overexpression of P-glycoprotein, overexpression of EVI1, a p53 alteration, DNMT3A mutation, FLT3 internal tandem duplication, a complex karyotype, decreased expression in BRCA1, BRCA2, or PALB2, or mutations in BRCA1, BRCA2, or PALB2.

20. (canceled)

21. (canceled)

22. A pharmaceutical composition comprising: i) an effective amount of a PARP inhibitor; ii) an effective amount of an antibody-drug conjugate of Formula (I): 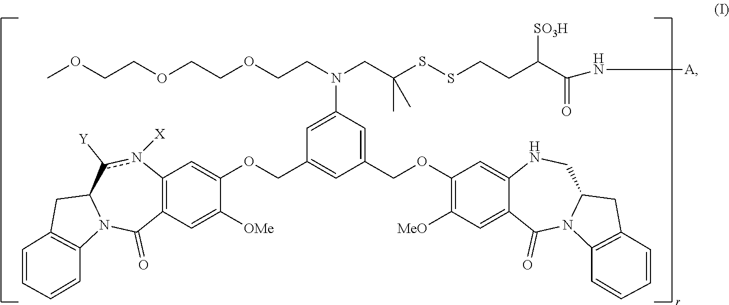
or a pharmaceutically acceptable salt thereof; and iii) a pharmaceutically acceptable carrier or diluent; wherein:
the double line [symbol] between N and C represents a single bond or a double bond, provided that when it is a double bond, X is absent and Y is hydrogen, when it is a single bond, X is hydrogen, Y is —SO₃H;
wherein A is the antibody or antigen-binding fragment thereof specifically binds to CD33 comprising a heavy chain variable region (VH) complementary determining region (CDR)1 sequence of SEQ ID NO:1, a VH CDR2 sequence of SEQ ID NO:2, and a VH CDR3 sequence of SEQ ID NO:3, and a light chain variable region (VL) CDR1 sequence of SEQ ID NO:4, a VL CDR2 sequence of SEQ ID NO:5, and a VL CDR3 sequence of SEQ ID NO:6; and
r is an integer from 1 to 10.

23. (canceled)

24. (canceled)

25. The pharmaceutical composition of claim 22, wherein the pharmaceutically acceptable salt is a sodium or potassium salt.

26. The pharmaceutical composition of claim 22, wherein the PARP inhibitor is a PARP-1 or a PARP-2 inhibitor.

27. (canceled)

28. The pharmaceutical composition of claim 22, wherein the PARP inhibitor is selected from the group consisting of 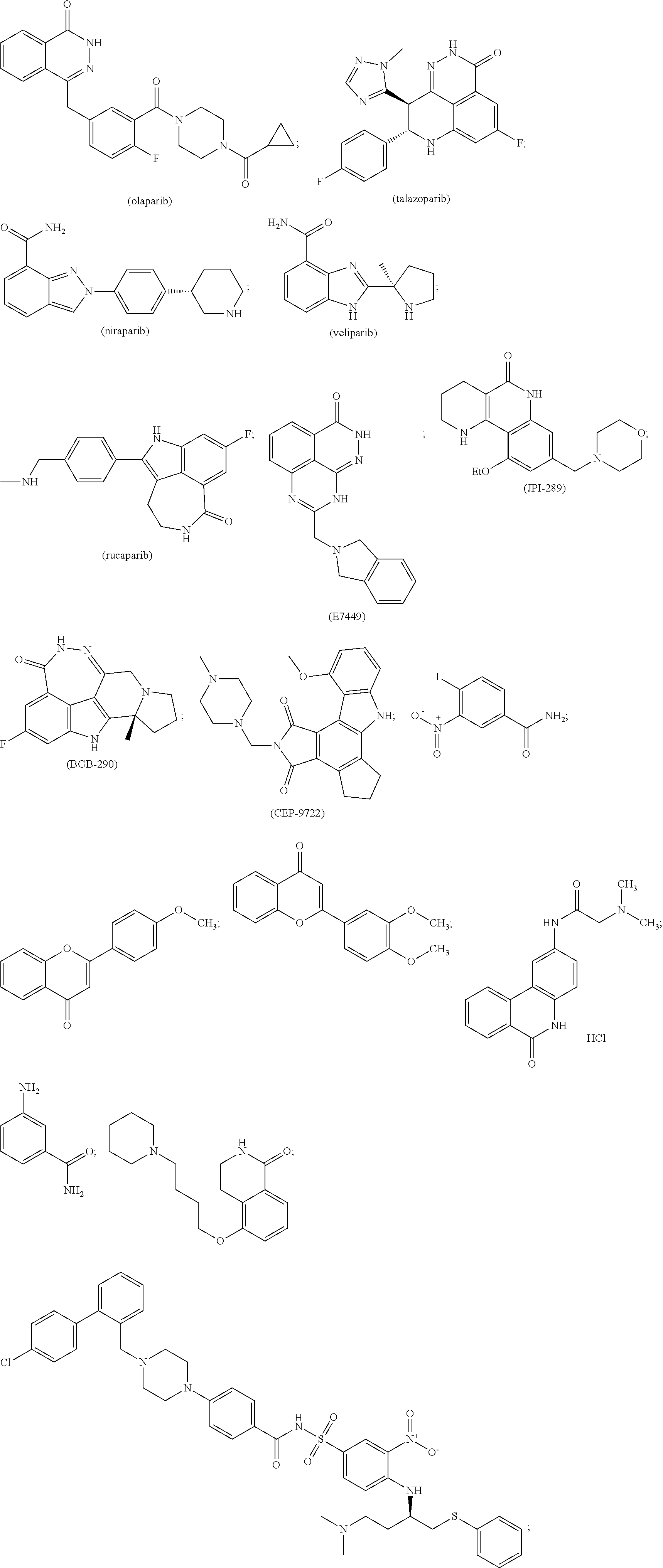
ABT767; and MP-124, or a pharmaceutically acceptable salt thereof.

29. (canceled)

30. The pharmaceutical composition of claim 22, wherein the antibody or antigen-binding fragment thereof comprises a heavy chain variable region comprising an amino acid sequence having at least 95% identity to the amino acid sequence of SEQ ID NO:7 or 9, and a light chain variable region comprising an amino acid sequence having at least 95% identity to the amino acid sequence of SEQ ID NO:8 or 10.

31. (canceled)

32. The pharmaceutical composition of claim 22, wherein the antibody or antigen-binding fragment thereof comprises a heavy chain variable region comprising the sequence of SEQ ID NO:9 and a light chain variable region comprising the sequence of SEQ ID NO:10.

33. The pharmaceutical composition of claim 22, wherein the antibody is huMy9-6.

34. (canceled)

35. (canceled)
